SEQUENCE LISTING (17 sequences)

SEQ ID NO: 1 - DNA, 20 bp, Artificial Sequence
  Primer used for amplification of the granule starch synthase promoter region from the vector pPGB121s
gattacgccaagctttaacg

SEQ ID NO: 2 - DNA, 32 bp, Artificial Sequence
  Primer used for amplification of the granule starch synthase promoter region from the vector pPGB121s
ggtttgtcgacgaaatcagaaataattggagg

SEQ ID NO: 3 - DNA, 21 bp, Artificial Sequence
  Oligonucleotide used for the production of DNA construct pPGB121s-B
tcgaccggtacctaggcccgg

SEQ ID NO: 4 - DNA, 21 bp, Artificial Sequence
  Oligonucleotide used for the production of DNA construct pPGB121s-B
gatcccgggcctaggtaccgg

SEQ ID NO: 5 - DNA, 26 bp, Artificial Sequence
  Oligonucleotide used for the production of DNA construct pGED
tcgacaagctttctagagcctcgagg

SEQ ID NO: 6 - DNA, 26 bp, Artificial Sequence
  Oligonucleotide used for the production of DNA construct pGED
gatccctcgaggctctagaaagcttg

SEQ ID NO: 7 - DNA, 47 bp, Artificial Sequence
  Oligonucleotide used for the production of the DNA construct pADAP
tcgaccggtaccaagcttgcgggctctagactcgagcctaggcccgg

SEQ ID NO: 8 - DNA, 47 bp, Artificial Sequence
  Oligonucleotide used for the production of the DNA construct pADAP
gatcccgggcctaggctcgagtctagagcccgcaagcttggtaccgg

SEQ ID NO: 9 - DNA, 12 bp, Artificial Sequence
  Oligonucleotide used for the production of DNA construct pPGB121s-B-B33
aattcaagcttg

SEQ ID NO: 10 - DNA, 12 bp, Artificial Sequence
  Oligonucleotide used for the production of DNA construct pPGB121s-B-B33
aattcaagcttg

SEQ ID NO: 11 - DNA, 20 bp, Artificial Sequence
  GBSSpro primer used for the amplification of the arabinanases encoding cDNA
acagctcaacaagtggtaac

SEQ ID NO: 12 - DNA, 55 bp, Artificial Sequence
  ARA-KDEL primer2 used for the amplification of the arabinanases encoding cDNA
gacttctcgagtggctggcctgttgtgaaggatgaactttagtctagaaatgctc

SEQ ID NO: 13 - DNA, 28 bp, Artificial Sequence
  ST SOE FWD primer used for the production of DNA construct pADA/ST-ARA
gacgaagcttatgattcataccaacttg

SEQ ID NO: 14 - DNA, 37 bp, Artificial Sequence
  ST SOE REV primer used for the production of DNA construct pADA/ST-ARA
ggagccggggttggcgtaggccactttctcctggctc

SEQ ID NO: 15 - DNA, 37 bp, Artificial Sequence
  ARA SOE FWD primer used for the production of DNA construct pADA/ST-ARA
gagccaggagaaagtggcctacgccaaccccggctcc

SEQ ID NO: 16 - DNA, 27 bp, Artificial Sequence
  ARA SOE REV primer used for the production of DNA construct pADA/ST-ARA
cagtctagactacacaacaggccagcc

SEQ ID NO: 17 - DNA, 10 bp, Artificial Sequence
  Oligonucleotide self-annealed and ligated into the EcoRI restricted pBI221 vector, creating the vector pBI221-2HindIII
aattaagctt

(19) United States
(12) Patent Application Publication
(10) Pub. No.: US 2003/0159178 A1
(43) Pub. Date: Aug. 21, 2003

(54) Method for remodelling cell wall polysaccharide structures in plants

(76) Inventors: Peter Ulvskov, Charlottenlund (DK); Henk Shols, Wageningen (NL); Richard Visser, Bennekom (NL); Bernhard Borkhardt, Farum (DK); Susanne Sorensen, Vallensbaek Strand (DK); Ronald Oomen, Zwolle (NL); Jean-Paul Vincken, Renkum (NL); Michael Skjot, Hedehusene (DK); Chantal Doeswijk Voragen, Wageningen (NL); Gerrit Beldman, Wageningen (NL)

Correspondence Address: MERCHANT & GOULD PC, P.O. BOX 2903, MINNEAPOLIS, MN, 55402-0903, US

(21) Appl. No.: 10/203,573

(22) PCT Filed: Feb. 12, 2001
(86) PCT No.: PCT/DK01/00093

(30) Foreign Application Priority Data
Feb. 10, 2000 (EP) 00610020.0

(51) Int. Cl. A01H 1/00; C12N 15/82; C12N 9/24

(52) U.S. Cl. 800/284; 435/200

(57) ABSTRACT

Methods for providing transgenic plants and parts hereof that, relative to the wild type state, is modified in a complex cell wall polysaccharide structure including pectins and hemicelluloses, the modification being in the overall glycosidic linkage pattern or the monosaccharide profile, comprising transforming a plant cell with a nucleotide sequence that causes an altered production of a complex cell wall polysaccharide-modifying enzyme such as endo-rhamnogalacturonan hydrolase, an endo-rhamnogalacturonan lyase, an endo-galactanase, an endo-arabinanase, an arabinofuranosidase, a galactosidase such as a beta-galactosidase, a xylosidase and an exo-galacturosidase. The modification can occur in vivo or post harvest, in which latter case the modifying enzyme is separated in the growing plant from its substrate, e.g. by targeting the enzyme to the Golgi, the endoplasmic reticulum or a vacuole, or is in a form that is inactive in the plant. After harvest the enzyme is brought into contact with its substrate or it is activated to provide the desired post harvest modification of the cell wall polysaccharide. The transgenic plant materials have improved functionalities and are useful in food and feed manufacturing and as pharmaceutically or medically active substances.

### FIELD OF THE INVENTION

The present invention relates to methods for remodelling the polysaccharide structure of the cell wall in higher plants by means of in vivo expression of polysaccharide-modifying enzymes.

### TECHNICAL BACKGROUND, PRIOR ART AND DISTINGUISHING FEATURES OF THE INVENTION

The cell wall of higher plants is comprised of cellulose, an interconnecting load bearing structure of mostly hemicellulosic polymers, pectic matrix polymers and globular and non-globular proteins. With the possible exception of the globular proteins, all polymer classes play structural roles in the cell wall. As the wall matures, cross links, both inter- and intra-chain links, are formed and eventually lignin is deposited in the cell wall rendering the cell wall quite stable and thus difficult to separate into its constituents, digest and process.

The present invention relates to gaining control in vivo over the structures of the complex cell wall polysaccharides. Of the wall polymers the simple, semicrystalline cellulose microfibrils are excluded from this class, as are the various categories of polymers which are built from non-sugar moieties, i.e. the proteins, lignin as well as suberin and waxes found in epidermal walls. Hemicelluloses and pectins sensu lato define the class of complex wall polysaccharides, although some of these may be referred to by a name indicating a narrower classification if particularly enriched or significant in a particular species or tissue, arabinogalactans in Larix wood and beta-glucan in cereal seeds to name a few examples. Excluded from the class of complex cell wall polysaccharides are, in the context of the present invention, those polysaccharides that are not structural components but serve other purposes, typically that of storage of carbohydrates to be remobilised during seed germination. Galactomannan as in guar, and xyloglucan as in tamarind are two examples.

Hemicellulosic polymers differ in type and abundance in, on one hand, plants belonging to the grasses, sedges and families closely related thereto and, on the other hand, the group of vascular plants. Carpita and Gibeaut (1993) refer to the non-grass type wall as a type-I cell wall, and the grass type cell wall as a type-II cell wall. Primary walls of type-I are characterized by roughly equal amounts of cellulose, hemicellulose and pectin. The predominant hemicellulosic polymer in undifferentiated cells is xyloglucan (not to be confused with the seed storage xyloglucan referred to above).

Xyloglucan is a minor hemicellulosic polysaccharide in type-II cell walls (˜5%), and also pectins are less abundant herein as compared to the type-I wall. A broad class of hemicellulosic polymers, i.e. xylans, arabinoxylans and glucans make up the remainder of the hemicellulosic polymers in the type-II wall. These hemicellulosic polymers are particularly relevant targets for the present invention when it is applied to remodel polysaccharide structures in type-II walls. For type-I cell walls, the present invention is particularly developed for the matrix polymers, i.e. pectins.

Pectins consist of two basic parts, i.e. an essentially unbranched polymer consisting of galacturonic acid residues (homogalacturonan, also known as the smooth region), and a polymer composed of alternating rhamnosyl and galacturonosyl residues, which can be substituted with long neutral side-chains (rhamnogalacturonan I, with “hairs”, also known as the hairy region). Pectic polysaccharides comprise between 30 and 50% of the cell walls of dicotyledonous plants (Carpita and Gibeaut 1993). The pectic matrix of plant cell walls is a complex mixture of homogalacturonan (HGA), rhamnogalacturonan I (RG-I) and rhamnogalacturonan II (RG-II) polymers (Voragen et al. 1995). HGA is an unbranched chain of 1,4 linked alpha-D-Galacturonic acid (GalA) residues that can be differentially methyl-esterified and/or acetylated. Relatively unesterified lengths of HGA (junction zones) can become Ca²⁺ cross-linked with similar regions of other HGA molecules to form polymers and higher-order structures that are capable of forming gels, whereas highly esterified HGA has a reduced potential for calcium promoted gelling. Gelling by other mechanisms often depends on a high methyl ester content. RG-I is a branched heteropolymer of alternating 1,2-alpha-L-Rhamnose (Rha) and 1,4-alpha-D-GalA residues (Lau et al 1985) that carry neutral side-chains of predominantly :1,4-beta-D-Galactose (Gal) and/or 1,5-alpha-L-Arabinose (Ara) residues attached to the rhamnose (Rha) residues of the RG-I backbone. They can either be single unit (beta-D-Galp-(1(4)), or polymeric such as arabinogalactan I and arabinan. Another type of arabinogalactan, arabinogalactan II, is mainly associated with proteins (arabinogalactan proteins). The branching pattern of the hairs is species-dependent. The side-chains of RG-I may be cross-linked to other pectic molecules by ester linkages through hydroxycinnamic acid residues such as ferulic acid (Fry 1986). It is generally accepted that homogalacturonan (HG) and rhamnogalacturonan I(RG-I) are covalently linked. However, the exact sequence of stretches of smooth and airy regions remains to be determined. The highly conserved RG-II molecule has a homogalacturonan backbone decorated with side-chains containing the richest diversity of sugars and linkages known, which may dimerise through a borate di-ester cross link (O'Neill et al. 1996).

Besides determining cell wall porosity, additional roles have been proposed for pectic polymers, including regulating cell-cell adhesion, cell expansion (McCann and Roberts 1994), wall mechanical properties (Chanliaud and Gidley 1997), as a source of signalling molecules (oligosaccharins) (Côtè and Hahn 1994) and involvement in cell differentiation and organogenesis (Satoh 1998).

The suitability of pectin for food applications is governed by many parameters, including its molecular weight, neutral sugar content, the proportion of smooth versus hairy regions, the degree of methyl and acetyl esterification, as well as the distribution of these ester groups along the homogalacturonan backbone (Daas et al. 1998, Braccini et al. 1999). For instance, cross-linking of homogalacturonans with Ca²⁺ is promoted when only small amounts of hairs are present, and consequently, gels with increased stability can be formed.

The primary structure of pectin from many abundant sources, e.g. potato tubers and sugar beets, is such that it is of an inferior quality for food applications when compared to e.g. apple or citrus pectin. In particular, the proportion of hairy regions of potato pectin is too high, and the degree of methyl esterification is too low (Ryden et al. 1990), the latter potentially caused by pectin methyl esterase acting post harvest. Additional problems with endogenous enzyme activities not withstanding, adjustments in the potato pectin structure are required to obtain satisfactory gelling properties, and to compete with higher-quality pectins.

Several pectic preparations with physiological effects on human cells and tissues have been extracted from plants, and biochemical interactions with macromolecular complexes of human origin have been demonstrated (Paulsen 2000). For example, it has been shown that compounds derived from hairy regions interact with the complement system in vitro, and it is thus inferred that these compounds are likely to act upon the immune system (immunomodulation) (Samuelsen et al. 1998, 1999, Yamada and Kiyohara 1999). However, many pharmaceutical claims are questioned due to poor characterization of the active preparations. Hence, lack of control over pectin structure has precluded determination of precise structure-activity relationships for these biochemical/physiological activities.

The concept of treating pectin and other plant cell wall polysaccharides with enzymes, either in the production of pectin or as a means of modifying the properties of polysaccharides to suit current and new applications, is an effort which has attracted considerable attention over the last 20 years. These enzymes include in particular glucanases, xyloglucanases, cellulases, fucosidases, xylanases, endo-polygalacturonases (endo-PGs or EPGs), pectin esterases, acetyl esterases specific to the different classes of polysaccharide decorated with acetyl groups, pectin lyases, exo-polygalacturonases and pectate lyases. Also the new class of rhamnogalacturonases, rhamnogalacturonan lyases, the galactanases, the arabinanases and the corresponding furanosidases have attracted a certain interest. Both alfa- and beta-specific enzymes are of interest. Using galactosidases as an example, some isoforms of alfa-galactosidases can mobilise galactomannan while the particular beta-specific variants can depolymerise pectic galactans. Similar considerations are relevant to virtually all classes of polysacchcaride modifying enzymes listed above. Many enzymes serving the purpose in nature, or selected by man for the modification of algal or microbial polysaccharides are also to be considered of interest with regard to higher plant cell wall polysaccharides due to shared motifs between polysaccharides of dissimilar composition and origin.

From the above it is clear that the polysaccharides in question, such as pectins, are extremely complex components of the cell wall, and there is a fairly good understanding of the fine structure of the individual polysaccharides. Considering the complexity of the polymers, it is not surprising that there are numerous enzymes that can be involved in their degradation, which in turn implies that a large “toolbox” is available for remodelling of these polymers.

However, the currently available concepts of modifying plant cell wall polysaccharides are predominantly based on post-harvest methods. This is the case with pectin which may be modified using post-harvest methods in which pectin is first extracted from the source plant material and only subsequently subjected to modification in order to result in modified pectin having desired characteristics for a given application.

In contrast, it would be advantageous to be able to modify cell wall polysaccharides in vivo, e.g. with reference to pectin, by modifying the structure of pectin, e.g. the structure of the hairy regions, since this would open up a range of different possibilities which are not currently available or feasible for technical and/or economic reasons. It is thus one major objective of the present invention to gain control over complex polysaccharide structure including pectin structure in living plants thereby obtaining plant materials containing remodelled cell wall polysaccharides having, relative to the parent plant, a new cell wall polysaccharide structure. As used herein, the terms “new polysaccharide structure and “new pectin structure” refer to polymers with a new arrangement and/or changed ratios of the monosaccharide building blocks of the polymer. When determining the linkage pattern as described hereinbelow, the quantitatively insignificant amount of new end-groups which result from a molecular weight down-shift (as seen e.g. during ripening) is disregarded, and polymers which are altered only in size, are not regarded herein as new structures. In determining both monosaccharide profile and linkage pattern, decoration with non-sugar substituents are also disregarded so that a polysaccharide which is different solely by virtue of e.g. increased methyl esterification, is not regarded a new structure. Accordingly, the present invention provides, in contrast to current technology, for in vivo generation of new complex cell wall polysaccharide structures as defined above.

In the following (and in the Examples below), the possibilities for modifying complex plant cell wall polysaccharide structures in vivo will be illustrated with reference to pectin structure using potato as an example. The invention is in no way limited to pectin or potato but it is useful in modifying a large range of cell wall polysaccharides and it is relevant to many crop species, and many plant organs in addition to tubers, but potato is an important crop in many countries, not only because it is consumed as such (boiled, baked, etc.) or after processing (French fries, chips, purees), but also because it produces a high-quality starch which can be used in many industrial applications. After extracting the starch from the potato tubers, substantial amounts of by-products (like fibre and proteins) remain, which have mainly found application in animal feed. However, these by-products contain constituents which have the potential of generating much higher value-products. The potato fibre fraction is a collection of various polysaccharides which together form the packaging material of the cell contents, i.e. the plant cell wall. Of these, pectin is probably the most interesting polymer because it is a known gelling agent in many food applications (Visser and Voragen 1996, Daas et al. 1998).

In accordance with the invention, particular focus is on the hairy regions of pectin rather than on, for instance, decoration of the homogalacturonan with ester groups. The amount of hairy regions determines the neutral sugar to uronic acid ratio. When this ratio is high, the water-binding capacity of the pectin is also high. This may be desirable in some food applications as well as non-food applications. When this ratio is low, the pectin structure approaches that of citrus pectins normally preferred by the food industry. Hence, lowering the neutral sugar-to-uronic acid ratio is important for the valorisation of pulp by-products from e.g. potato starch and beet sugar production.

Accordingly, another important objective of the present invention is to decrease the proportion of hairy regions of pectins in planta by genetic modification. In addition to an improvement of the gelling characteristics of potato pectin, this should also facilitate the starch extraction process, resulting in a higher starch yield.

Another objective is to tailor and recover pectin hairy regions for their use in low viscosity food products (e.g. drinking yoghurt and similar diary products). The particular embodiments of the invention referred to as “self-processing” tubers (or self-processing plant material in general) provides additional technology to meet this demand. “Self processing” refers to the property that the plant tissue, upon disruption post harvest, releases enzymes stored intracellularly. The released enzyme catalyses a modification of the cell wall material, typically excision of the poly- or oligosaccharides of interest for easy recovery. This technology can also be applied to medical uses of tailored pectic polymers and oligomers derived from these.

In contradistinction to post-harvest modification of pectic polymer structure which makes use of degrading enzymes, in vivo modification can also make use of genes encoding biosynthetic enzymes or components of synthase complexes. It will be appreciated that up or down-regulation of the activity of the synthases or decorative enzymes will broaden the engineering options. A few decorative transferases have been identified and may turn out to be valuable in this regard (Perrin et al. 1999, Edwards et al. 1999, WO 99/60103). Such enzymes are candidates for pectin modification according to the present invention.

Some enzymes involved in the interconversions of nucleotide sugars are known, and based on characterized mutants (see below) it is contemplated that genes encoding these enzymes may be useful for indirect manipulation of biosynthesis of pectin and other cell wall polysaccharides. However, presently the genes encoding polysaccharide-modifying enzymes already demonstrated to be useful for post-harvest pectin modification are among preferred tools for modifying pectin in vivo.

Several mutant Arabidopsis displaying a modified cell wall phenotype have been isolated. Some of these are affected at the level of nucleotide sugar pools. Only one of the existing range of cell-wall mutants, mur 8, is likely to be a specific pectin mutant, as it is deficient in rhamnose (Reiter et al. 1997), while mur 1 (deficient in fucose), and mur 4 (reduced in arabinose) (Mayer 1998) are likely to be affected in pectins among other cell wall polysaccharides. However, such mutants have been generated in a random fashion, which is manageable in Arabidopsis but not in crop plants in which cell wall polysaccharide modification is relevant.

WO 91/08299 discloses a process for the inhibition of the production of a gene product in a plant cell using antisense technology with the objective of controlling fruit ripening. Target genes include pectin esterases, galactosidases, glucanases and xylanases. Accordingly, this disclosure is not concerned with in vivo generation of new complex cell wall polysaccharides as provided herein.

WO 99107857 discloses nucleic acids encoding pectate lyase from plants and transgenic plants and parts and progeny hereof having altered pectate lyase activity. Although it is evident that increased pectate lyase activity in a plant may lead to altered pectins, this alteration is only in the sense that molecular weight changes of the pectic polymers may occur which, however, does not lead to a new pectin structure as defined herein.

WO 93/13212 discloses DNA encoding a pectin esterase iso-enzyme for transformation of e.g. tomato to reduce pectin esterase activity.

A poster entitled “Modification of pectin by expression of a fungal endo-beta-1,4-galactanse in potato” presented by Sørensen et al. (1999) suggests that it might be desirable to modify the hairy regions of potato pectin by reducing the amount of linear galactans attached to rhamnose residues of RG-I, and that it is expected that these galactans can be degraded by a fungal endo-galactanase from Aspergillus aculeatus. The poster reports that transgenic plants expressing the galactanase gene in leaves and tuber under control of the Granule Bound Starch Synthase (GBSS) promoter or the patatin promoter were produced, but possible effects of the galactanase expression on the cell wall polysaccharides was not addressed neither theoretically nor by experiment.

Thus, the prior art suggests that is possible to control ripening processes in the plant cell wall, and that these manipulations can change molecular weight distribution of polymers and decorations with substitutions such as methyl esterification. It is not surprising that this gaining control over ripening is possible in as much as ripening is a natural process in plants which is already subject to regulation with regard to the speed with which it progresses. In the present context, however, such changes are not regarded as being accompanied by new cell wall polysaccharide structures.

Some workers have proposed rather unspecified downstream effects of gene expression on cell wall properties, but no specific technology has been developed. Knowing that plants actively and in a carefully regulated manner metabolise their cell walls during development, it is very surprising that it can be tolerated by the plant to re-engineer the cell wall beyond merely accelerating or slowing down the natural progression of wall metabolism. However, a clear distinction must be made between the rate changes of wall metabolism which are associated with delay of ripening as demonstrated in the prior art where polymer esterification and molecular weight changes predominate while polymer composition largely stays the same, and the achievement of the present invention which provides a novel technology for a targeted tailoring of complex cell wall polysaccharides, affording a highly predictable change in the monosaccharide profile and/or the overall linkage pattern as determined e.g. by methylation analysis.

Accordingly, the present invention relates to novel technology for targeted, specific remodelling or modification of complex plant cell wall polysaccharides. Generally, benefits from this technology fall in four broad categories:

1. Improving Agronomic Properties of Crop Plants

Modified plant architecture. Certain selective interferences with the course of cell wall metabolism will affect cell differentiation, morphology and development. Self-pruning plants for example may be engineered by modifying cell wall components which are critical for determining cell fate and course of development.

Fertility. Where cell separation is a critical developmental process as in seed dispersal (WO 97/13865 ), pollen dispersal and growth of the pollen tubes along the style middle lamellae, the course of development may be altered through cell wall modifications.

Pathogen invasion. Pathogenic fungi and bacteria invade plant tissues through the orchestrated deployment of a set of polysaccharide degrading enzymes which match the architecture of the host cell wall. The present technology provides the means for altering the cell wall so as to retard the penetration. As signalling molecules, the so-called oligosaccharins, are released from the wall during pathogen invasion, a new cell wall architecture may result in a different pattern of oligosaccharin release from the wall, and hence alter the course of pathogenesis. Reduced symptom development will be achieved.

2. Improving Plant Products

Dietary fibre. Wall modifications may be used to increase the abundance of dietary fibre in food products, and alter the ratio between insoluble and soluble dietary fibres, most often in favour of the latter with which many health claims are associated.

Texture. Cell walls are responsible for vegetable and fruit texture, and plays a role in the textural changes which accompany storage. Both texture and shelf life will thus be subject to modification using the current invention. As it pertains to shelf life the technology presented here complements control of ripening (speed up or delay) by transgenic modification as described in the prior art.

Material properties. Structural properties of wood and fibres rely on overall plant anatomy, fibre cell geometry, secondary wall formation, lignification and polysaccharide polymer make up. Plant material properties originating in the latter may be improved through the targeted cell wall engineering made possible by the present invention. Properties of relevance for medical materials, hydrocolloids for coating of implants and some medical instruments, include water binding capacity. Water binding capacity can be controlled through modifications of e.g. pectin structure as enabled by the present invention (see also “Tailored pectins” heading 4 below).

Added value to by-products. Where pulp from paper or fibre manufacturing, from breweries, from production of starch or sugar represents a rich source of complex plant polysaccharides which do not fulfil criteria for a particular industrial use, the present invention may be employed for the engineering of the crop plant in question so as to valorise the pulp by making it a source of higher value-polysaccharides. One example hereof is targeting the valorisation at the pectic fraction as exemplified in the following. Functional feed cell wall composition largely determines digestibility and hence nitrogen use efficiency in livestock and poultry. Notably in non-ruminant animals, young piglets for example and in poultry the cell wall polysaccharide digestibility is a limiting factor in respect of efficient feed utilisation. Application of the novel technology presented herein can lead to feed products with superior digestibility, and hence improved growth rates in the animals. In ruminant animals increased digestibility of polysaccharides and cell wall components will affect the protein degradation in the rumen, because the microorganisms in the rumen will utilise the released saccharides instead of protein and in this way less protein is degraded in the rumen and less ammonia is produced and in this way increased digestibility of the polysaccharides and cell wall components will save proteins from degradation in the rumen whereby more protein components is taken up in the intestines. In this way improved digestibility of the cell wall components and polysaccharides will increase the utilisation of protein and decrease the excretion of ammonia or nitrogen from rumen degraded protein.

3. Improving Processing Characteristics

Beverage filtering properties. Liquefication of plant materials is applied in juice and wine manufacturing, e.g. using food industrial enzymes so as to increase yield and retard clotting of filters. The present invention is useful with respect to complement or supplant these practices and to decrease/remove certain polysaccharide fractions that cause problems during such processes.

Malting properties. During malting, starch is degraded whereas the beta-glucan of the specialised cell wall of the caryopsis is only solubilised and may cause problems with regard to beer clarity. While most biotechnological approaches to this problem has been directed at engineering the yeast to be able to fully digest the beta-glucan, the present invention can be employed to improve. the processing characteristics of the beta-glucan component of the cell walls and in general to improve the processing during malting and also to improve the end products.

Baking properties. Rheological properties of doughs are often modified using technical enzymes rich in xylanase and glucanase activities acting upon the cell wall polysaccharides. Starting materials in which the cell wall polysaccharides have already been modified as desired using the present invention, will be able to complement or supplant the practice of adding technical enzymes and in addition, other modifications of the polysaccharides and cell wall components will improve the properties of the dough and improve the baking properties.

Fibre retting. Fibres, e.g. of flax, are isolated either using treatment with industrial enzymes or through a classical “fermentation” process known as retting. Either may be supplanted or complemented using fibre plants in which the fibres themselves, or the tissue in which they are embedded, are modified with regard to cell wall properties as enabled by the present invention. In addition, improvement (including the processing) of other plant fibres (cotton, hemp etc.) employed as textiles can also be achieved using the technology of the invention.

4. Isolated Polysaccharides.

Tailored pectins as food ingredients and additives. Designed pectin, e.g. from plant materials normally containing pectins of inferior quality as described above can be obtained using the present invention. Generally, benefits from such technology may include maintenance of the physiological activity of pectin at low viscosity, uniformity of ester distribution, production of high molecular weight pectin with low viscosity, elimination of acid from waste water and manufacture of low ester pectin with no loss in molecular weight. Other benefits include simpler processing, high yield, high strength, utilisation of inexpensive raw materials, moderate process conditions, ease of recovery, low cost, low environmental impact of processing and higher quality. In addition, the present technology will also make use of non-refined (e.g. produced without most of the traditional extraction procedures) products possible as the technology makes it possible to remove undesired polysaccharide and cell wall components involved in e.g. the creation of non-palatable appearance and to improve the quality of “ingredients” of low quality components.

Tailored pectins as medical materials and pharmaceuticals. Bioactive poly- and oligosaccharides derived from cell wall polysaccharides including pectins have been detected in several plant species, many of which are exotic plants. The present invention will provide significant control over cell wall polysaccharide polymer structure, and allow poly- and oligosaccharides of e.g. crop plants to be designed and thus rendered bioactive. Benefits include high efficiency and low cost in processing using well known processing steps at e.g. sugar factories, starch factories, breweries etc., optimised for one or more particular crop plants.

### SUMMARY OF THE INVENTION

In a first aspect the invention pertains to a method for providing a transgenic plant material that, relative to the wild type state, is modified in a complex cell wall polysaccharide structure, the modification being at least one of the overall glycosidic linkage pattern and the monosaccharide profile, the method comprising the steps of: (i) providing a nucleic acid construct comprising a nucleotide sequence, which, following the introduction of the construct into a plant cell, results in an altered production of at least one target cell wall polysaccharide-modifying enzyme, (ii) transforming a plant cell with the nucleic acid construct, and (iii) deriving from said transformed plant cell a plant cell culture, a plant tissue or a transgenic plant in which the production of the at least one cell wall polysaccharide-modifying enzyme is altered to obtain transgenic plant cells, plant tissues or plants in which the targeted substrate cell wall polysaccharide, relative to the wild type plant, is modified to have a monosaccharide profile where the proportion of at least one monosaccharide is changed by at least 10% or the proportion of at least one glycosidic linkage is changed by at least 10%.

In one embodiment hereof the nucleotide sequence, which, following the introduction of the construct into a plant cell, results in an altered production of at least one target cell wall polysaccharide-modifying enzyme is a sequence coding for such an enzyme, such as a cell wall polysaccharide-modifying enzyme of fungal or microbial origin, that is capable of modifying the targeted cell wall polysaccharide including an embodiment wherein the coding sequence is operably linked to a promoter directing the expression of the coding sequence which promoter e.g. is of plant origin such as a plant tissue or organ specific promoter including a storage organ specific promoter. In this context, an interesting promoter is a promoter that is capable of directing expression of the cell wall polysaccharide-modifying enzyme in potato tubers including a promoter selected from the group consisting of the granule bound starch synthase (GBSS) promoter and the B33 promoter.

In a further embodiment the at least one target cell wall polysaccharide-modifying enzyme, the production of which is altered, is an endogenous enzyme, i.e. an enzyme naturally produced in the transformed plant cell, including a particular embodiment wherein the nucleotide sequence, which, following the introduction of the nucleic acid construct into a plant cell results in an altered production of at least one target cell wall polysaccharide-modifying enzyme, is a sequence that modulates the expression of an endogenous sequence coding for an enzyme that is capable of modifying the targeted cell wall polysaccharide. As one example of such an embodiment, the sequence that modulates the expression of an endogenous sequence coding for an enzyme that is capable of modifying the targeted cell wall polysaccharide, is a sequence coding for an antisense sequence that reduces the expression of a repressor or reduces the production of an inhibitor of the endogenous target cell wall polysaccharide-modifying enzyme. In another example, the sequence that modulates the expression of an endogenous sequence coding for an enzyme that is capable of modifying the targeted cell wall polysaccharide, is a sequence that, following a recombination event, causes the insertion of a new or modified promoter operably linked to the endogenous target cell wall-modifying enzyme, said promoter is capable of directing the expression of the coding sequence for the cell wall-modifying enzyme.

In another embodiment of the above method, the nucleotide sequence, which, following the introduction of the construct into a plant cell results in an altered production of at least one target cell wall polysaccharide-modifying enzyme, is a sequence that, following a recombination event, causes the expression of an endogenous sequence coding for an enzyme that is capable of modifying the targeted cell wall polysaccharide to be targeted to a location in the plant cell where such enzyme is not normally present.

In useful embodiments of the above method, the nucleic acid construct is a viral vector that, following introduction into a plant cell, is not integrated into the genome of the cell.

In specific embodiments of the above method the targeted complex cell wall polysaccharide which is modified by a cell wall polysaccharide-modifying enzyme is a pectin or a hemicellulosic polysaccharide. Suitable pectin- or hemicellulose-modifying enzymes include endo-rhamnogalacturonan hydrolases, endo-rhamnogalacturonan lyases, endo-galactanases, endo-arabinanases, arabinofuranosidases, galactosidases such as beta-galactosidases, xylosidases and exo-galacturonases and orthologs or isoforms hereof.

In a still further embodiment of the above method the nucleotide sequence, which, following the introduction of the construct into a plant cell, results in an altered production of at least one target cell wall polysaccharide-modifying enzyme, is sufficiently different from endogenous genes of the host plant so that co-suppression will not occur.

In yet another embodiment of the above method, an auxiliary enzyme, including as examples a methyl esterase, an acetyl esterase or a glycosidase that removes single monosaccharides from polymers, such as e.g. an arabinofuranosidase, a galactosidase, a xylosidase or a fucosidase, is co-expressed with the target cell wall polysaccharide-modifying enzyme, wherein co-expression facilitates access of the polysaccharide-modifying enzyme to its substrate.

In a further aspect the present invention relates to a method for modifying the biosynthesis in a plant cell of at least one complex cell wall polysaccharide to obtain a transgenic plant material that, relative to the wild type state, is modified in a complex cell wall polysaccharide structure, the modification being at least one of the overall glycosidic linkage pattern and the monosaccharide profile. This method comprises the following steps: (i) providing a nucleic acid construct comprising a nucleotide sequence, the expression of which in a plant cell results in that at least one cell wall polysaccharide-modifying enzyme is targeted to a compartment in the plant cell where it is not normally present or in that the expression of a polypeptide naturally produced in the plant cell and affecting the biosynthesis of a cell wall polysaccharide is changed, (ii) transforming a plant cell with the nucleic acid construct, and (iii) deriving from said transformed plant cell a plant cell culture, a plant tissue or a transgenic plant in which the at least one cell wall polysaccharide-modifying enzyme, relative to the wild type plant, occurs in a different compartment.

The above method includes an embodiment wherein the obtained transgenic plant cells, plant tissues or plants in which the targeted substrate cell wall polysaccharide, relative to the wild type plant, is modified to have a monosaccharide profile where the proportion of at least one monosaccharide is changed by at least 10% or the proportion of at least one glycosidic linkage is changed by at least 10%.

In further specific embodiment of the above method the at least one cell wall polysaccharide-modifying enzyme is targeted to the Golgi apparatus including membrane vesicles fusing with or budding off from the Golgi stacs including an embodiment wherein the nucleotide sequence, the expression of which in a plant cell results in that at least one cell wall polysaccharide-modifying enzyme is targeted to the Golgi apparatus, is a sequence coding for a chimeric gene product comprising the at least one cell wall polysaccharide-modifying enzyme and a sequence capable of targeting the chimeric gene product to the Golgi. Such a targeting sequence is e.g. a type II membrane anchored Golgi protein, e.g. a sialyl transferase, a N-acetylglucosaminyl-transferase, a fucosyl transferase, a xylosyl transferase or a galactosyl transferase including a fragment thereof, or a soluble Golgi targeted protein such as e.g. Pisum sativum reversibly glycosylatable polypeptide (RGP1) or a fragment thereof.

In another specific embodiment of the above method for modifying the biosynthesis of cell wall polysaccharides the nucleotide sequence, the expression of which in a plant cell results in that at least one cell wall polysaccharide-modifying enzyme is targeted to a compartment in the plant cell where it is not normally present, is operably linked to a promoter in the genome of the plant cell into which it is introduced.

In another aspect the present invention pertains to a method for providing a transgenic plant comprising parts in which at least one complex cell wall polysaccharide, such as pectin or a hemicellulosic polysaccharide, can be enzymatically processed after harvest by an enzyme present in the plant material itself, i.e. a “self-processing” plant, the method comprising: (i) providing a nucleic acid construct comprising a nucleotide sequence, which, following the introduction of the construct into a plant cell, causes a cell wall polysaccharide-modifying enzyme to be expressed in a non-apoplastic or non-Golgi compartment of the plant cell or the expression of a cell wall polysaccharide-modifying enzyme in a form that is inactive under in vivo conditions but can be activated following harvest of plant material derived from the plant cell, (ii) transforming a plant cell with the nucleic acid construct, and (iii) deriving from said transformed plant cell a transgenic plant material in which, under appropriate post harvest conditions, the at least one complex cell wall polysaccharide can be enzymatically processed after harvest by bringing the at least one complex cell wall polysaccharide into contact with the cell wall polysaccharide-modifying enzyme that is expressed in a non-apoplastic or non-Golgi compartment or by subjecting the harvested plant material to conditions under which the enzyme being expressed in an in vivo inactive form is activated.

In one specific embodiment hereof, the nucleotide sequence, which, following the introduction of the nucleic acid construct into a plant cell, causes a cell wall polysaccharide-modifying enzyme to be expressed in a non-apoplastic or non-Golgi compartment is a sequence that causes the cell wall polysaccharide-modifying enzyme to be targeted during growth of the plant to a cell compartment selected from the group consisting of a vacuole, the endoplasmic reticulum, the cytoplasm and a plastid, including embodiments wherein the cell wall polysaccharide-modifying enzyme caused to be expressed in a non-apoplastic or non-Golgi compartment is encoded by a sequence comprised in the nucleic acid construct that is introduced into the plant cell or by an endogenous sequence present in the genome of the cell into which the nucleic acid construct is introduced.

In useful embodiments of the above method for providing post harvest self-processing plants, the cell wall polysaccharide-modifying enzyme is selected from the group consisting of an endo-polygalacturonase, an endo-pectin lyase, a pectate lyase, a rhamnogalacturonan hydrolase, a rhamnogalacturonan lyase, an endo-glucanase, an endo-xylanase and an isoform or ortholog hereof. In useful embodiments, the expression of such enzymes is directed by a plant promoter.

In specific embodiments of the above method where the cell wall polysaccharide being processed after harvest is pectin, the processing of pectin is in the regions between rhamnogalacturonan and homogalacturonan regions.

In the method of providing post harvest “self processing” plants, one useful embodiment is one wherein the plant cell is further transformed with a nucleic acid sequence causing an enzyme that is capable of in vivo modifying the structure of at least one complex cell wall polysaccharide, including a cell wall polysaccharide that can be enzymatically processed after harvest by an enzyme present in the plant material itself, to be expressed. Such a nucleic acid sequence is e.g. a sequence coding for a cell wall polysaccharide-modifying enzyme, or a sequence coding for a product that affects the expression of an endogenous sequence coding for a cell wall polysaccharide-modifying enzyme. In useful embodiments, the cell wall polysaccharide-modifying enzyme is targeted to the apoplast.

Useful enzymes that are capable of in vivo modifying the structure of at least one complex cell wall polysaccharide include endo-rhamnogalacturonan hydrolases, endo-rhamnogalacturonan lyases, endo-galactanases, endo-arabinanases, arabinofuranosidases, galactosidases such as beta-galactosidases, xylosidases and exo-galacturonases and orthologs or isoforms hereof.

In a still further aspect the invention relates to a method of providing a plant cell wall polysaccharide material having, relative to the wild type state, a modified structure and composition, the method comprising the steps of: (i) providing transgenic plants, e.g. potato plants, using the above method of providing post harvest “self-processing” plants, (ii) cultivating and harvesting said plants and isolating herefrom parts in which at least one complex cell wall polysaccharide, such as e.g. pectin, can be enzymatically processed after harvest by an enzyme present in the plant material itself, (iii) subjecting said parts to conditions where the cell wall polysaccharide-modifying enzyme expressed in a non-apoplastic or non-Golgi compartment is brought into contact with its cell wall polysaccharide substrate or the cell wall polysaccharide-modifying enzyme expressed in a form that is inactive under in vivo conditions becomes activated to obtain a modified cell wall polysaccharide, and (iv) isolating the modified cell wall polysaccharide material.

In useful embodiments of this method the modified cell wall polysaccharide in the material as obtained is modified to have a monosaccharide profile where the proportion of at least one monosaccharide is changed by at least 10% or the proportion of at least one glycosidic linkage is changed by at least 10%.

In a further aspect the invention provides a plant cell wall polysaccharide material having, relative to the wild type state, a modified structure and composition, which material is obtained by the above method.

There is also provided a transgenic plant or progeny of the plant or part thereof obtained by the method according to the first aspect of the invention for providing a transgenic plant material that, relative to the wild type state, is modified in a complex cell wall polysaccharide structure and plant cell wall polysaccharide-containing materials obtained from such transgenic plants or progeny or parts including such transgenic plant materials which, relative to a material containing the corresponding wild type cell wall polysaccharide, has at least one altered functional characteristic such as an altered pharmaceutical activity, water binding capacity, processibility, gelling property, thickening property and digestibility.

In yet other aspects the invention pertains to the use of the above transgenic plants or progeny of the plants or parts thereof or plant cell wall polysaccharide-containing material in the manufacturing of food products, food additive products, feed products, pharmaceutical or medical products and cosmetic products and pharmaceutical or medical products comprising the above plant cell wall polysaccharide-containing material, including as examples pharmaceutical compositions, implant materials, medical devices and surgical adhesives.

There is also provided a transgenic plant or progeny of the plant or part thereof obtained by the above method for modifying the biosynthesis in a plant cell of at least one complex cell wall polysaccharide and plant cell wall polysaccharide-containing materials obtained from such transgenic plants or progeny or parts including such transgenic plant materials which, relative to a material containing the corresponding wild type cell wall polysaccharide, has at least one altered functional characteristic such as an altered pharmaceutical activity, water binding capacity, processibility, gelling property, thickening property and digestibility.

In yet other aspects the invention pertains to the use of the above transgenic plants or progeny of the plants or parts thereof or plant cell wall polysaccharide-containing material in the manufacturing of food products, food additive products, feed products, pharmaceutical or medical products and cosmetic products and pharmaceutical or medical products comprising the above plant cell wall polysaccharide-containing material, including as examples pharmaceutical compositions, implant materials, medical devices and surgical adhesives.

In a still further aspect there is provided a method of producing a material comprising a complex plant cell wall polysaccharide having, relative to the corresponding cell wall polysaccharide in the wild type state, a modified structure and/or a modified composition, the method comprising the steps of: (i) providing a cultivatable transgenic plant, such as e.g. a potato plant, using the method according to the first aspect of the invention or the above method for modifying the biosynthesis in a plant cell of at least one complex cell wall polysaccharide, or a cultivatable progeny hereof, (ii) cultivating said transgenic plant or progeny under appropriate plant cultivation conditions to obtain a plant material comprising at least one complex cell wall polysaccharide having a modified structure and/or modified composition, and (iii) isolating from the cultivated plants the material comprising the modified cell wall polysaccharide. In useful embodiments of this method the material isolated from the cultivated plants comprises a cell wall polysaccharide that, relative to the wild type plant, is modified to have a monosaccharide profile where the proportion of at least one monosaccharide is changed by at least 10% or the proportion of at least one glycosidic linkage is changed by at least 10%.

### DETAILED DISCLOSURE OF THE INVENTION

Throughout the following description and in the appended claims several general and specific terms and expressions will be used to define the invention. Below such terms and expressions will be defined and explained:

The terms “new polysaccharide structure” and “new pectin structure” designate polymers with a new glycosidic linkage arrangement and/or changed ratios of the monosaccharide building blocks of the polymer, i.e. a changed monosaccharide profile. When determining the linkage pattern, the quantitatively insignificant amounts of new end-groups which result from a molecular weight down-shift (as seen e.g. during ripening) is disregarded, and polymers which are altered only in size, are not regarded as new structures. For both monosaccharide profile and linkage pattern determination, decoration with non-sugar substituents are also disregarded so that a polysaccharide which is different solely by virtue of e.g. increased methyl esterification, is not regarded as a new structure. Monosaccharide profile of a polysaccharide, or polysaccharide mixture, is determined by gas-chromatography of e.g. aditol acetate derivatives of polysaccharide hydrolysates, or by HPAEC with or without separate colourimetric determination of uronic acids. Linkage analysis is usually performed by removal of non-sugar decorations of the poly- or oligo-saccharides followed by thoroughly methylating all free hydroxyl groups prior to hydrolysis and derivatisation for gas chromatography. Both for determination of monosaccharide profile and linkage pattern a less than complete starch removal may skew the results, and the experimenter is required to assess this non-random source of variation. Monosaccharide profile is a property with both quantitative and qualitative components to it, while linkage pattern is a largely qualitative trait. In particular, quantitative traits are subject to biological variation. The present invention allows for the introduction of new structures as defined herein in complex cell wall polysaccharides, and these are recognised by comparison with a wild type control (defined below). A transformant is recognised as being different from the wild type state if the monosaccharide profile differs in one monosaccharide by at least 10 mol % including at least 15 mol %, at least 20 mol % or at least 25 mol %, or if it differs, relative to the wild type plant, in linkage pattern analysis by at least 10 mol % including at least 15 mol %, at least 20 mol % or at least 25 mol % in at least one residue. Simultaneous changes in both monosaccharide profile and linkage pattern are not required but may occur. Some conceivable modifications regard arrangement alone and is manifested solely in a novel linkage pattern while others are purely compositional modifications in which only the monosaccharide profile is changed.

The term “transgenic” or “transformed” plant refers to a plant which by the process of genetic transformation is made to contain nucleic acid sequences, including DNA-methylation patterns, which are not normally present in the plant, or nucleic acid sequences which are in addition to the sequences which are normally present in the plant, or DNA sequences which are normally present in the plant but which are altered compared to the native sequence. In the present context, alterations also include changes in their DNA-methylation pattern or changes in placement in the genome.

The term “wild type” refers to a plant which is neither stably transformed nor manipulated to transiently express extraneous genetic material. Wild type plants are used to gauge biological variation and thus by comparison allow for identification of manipulated plants which display a genuinely new phenotype. Wild type control plants should be of the same cultivar as the modified plant under investigation and should be grown under essentially identical conditions to serve as a proper control in the context of the present invention. Organs and tissues for control analysis shall be sampled from experimental and control plants at similar developmental stages.

The term “nucleic acid” refer to any nucleic acid substance including DNA, RNA, LNA (locked nucleic acids), PNA, RNA, dsRNA, RNA-DNA-hybrids that is capable of changing the maintenance or inheritance of the genetic material and/or the DNA-methylation pattern in the plant. Also included are nucleic acids comprising non-naturally occurring nucleosides.

The term “nucleic acid construct” refers to a genetic sequence used to transform plant cells to generate progeny transgenic plants. A nucleic acid construct comprises at least a coding region for a desired gene product, operably linked to the 5′ and 3′ regulatory sequences for the expression in plants. Such constructs may be chimerc, i.e. consisting of a mixture of sequences from different sources, or non-chimeric. The orientation of the coding region may be either of a sense or antisense orientation, depending on the intended function of the gene product in question.

Transformation by homologous recombination has recently been proved possible in higher plants (Kempin et al. 1997). It is reasonable to expect this to change and pave a way for preparing transgenic plants according to the above definition. Accordingly, the definition of “nucleic construct” is to be understood as encompassing these new technologies. Particular technical approaches to implementing the present invention using homologous recombination include, but is not limited to: Recruitment of endogenous cell wall polysaccharide-modifying enzymes by replacement of the promoter sequence or by knock-out of a repressor. This may be generally acceptable to the plant, or at least acceptable where the gene in question exists in more than one isoform. Following the same rationale, subcellular targeting of an endogenous gene product can be changed by replacement of its signal sequence. Finally, heterologous genes in toto or their coding regions can be introduced in the plant genome through replacement of an endogenous gene.

Also, the use of e.g. plant viruses for carrying foreign genetic material into plants and thus modify gene expression in infected cells is feasible, and may indeed be an attractive route for genetic modification of plants without producing stably transformed plants. Mature plants can be infected shortly before harvest and modified tissue recovered as soon as the desired phenotype has developed. When direct utilisation of transformed cells are referred to herein, it is these transiently modified cells that are alluded to.

The term “antisense” refers to the sequence of a DNA strand that is complementary to the sequence of the sense strand and that cannot be translated into the polypeptide encoded by the structural gene. For purposes of the present invention, antisense refers to a nucleic acid construct that is operably linked to a promoter with all or part of the sequence in reverse orientation so that following transcription into an RNA molecule, hybridisation can occur between sense and antisense sequences thereby leading to a reduced level of the polypeptide in question. The term “sense” as used herein refers to the sequence of the DNA strand of a structural gene that is transcribed into an mRNA molecule copy which is then translated into the polypeptide encoded by the structural gene.

The term “operably linked” means that the regulatory sequences which are necessary for the expression of the coding sequence are placed in the nucleic acid molecule in the appropriate position relative to the coding sequence so as to effect the expression of the coding sequence. Alternatively, the coding sequence can be operably linked to regulatory sequences in the genome of the cell which is transformed with the coding sequence. The term “promoter” as used herein refers to a DNA sequence which causes, or is needed for transcription of DNA into an RNA molecule. The promoter may be a tissue specific or organ specific promoter, a promoter which is active at specific developmental stages or it may be an inducible promoter, e.g. one of the inducible promoters discussed below or another inducible promoter known in the art, or a constitutive promoter such as the cauliflower mosaic virus 35S promoter or another constitutive promoter known in the art.

The term “vector” means a nucleic acid molecule that is capable of replicating in a cell (or capable to be multiplied in vitro, e.g. by PCR methods) and to which another nucleic acid sequence can be operably linked so as to bring about replication of the attached nucleic acid sequence. Commonly used vectors are discussed below and include bacterial plasmids and bacteriophages.

The nucleic acid constructs as defined above can be incorporated into plant cells using conventional recombinant nucleic acid technologies. Generally, such techniques involve inserting nucleic acid in an expression vector which contains the necessary elements for the transcription and translation of the inserted protein coding sequence and one or more marker sequences to facilitate selection of transformed cells or plants. Once the nucleic acid construct has been cloned into an expression vector, it may be introduced into the plant cell using conventional transformation procedures known by a person skilled in the art. These include, but are not limited to, use of Agrobacterium vectors such as A. tumefaciens and A. rhizogenes, PEG treatment of protoplast, biolistic DNA delivery, bombardment with gold particles coated with the nucleic acid construct, electroporation of protoplast or direct nucleic acid uptake as generally described in Plant Tissue Culture Manual (Lindsey, 1992, Kluwer Academic Pubs., Dordrecht). It will be apparent to persons skilled in the art that suitable methods of transformation in any given case depend upon the plant to be transformed. As used herein, the term “transformation” refers to the event of introduction of a nucleic acid into a plant cell irrespective of whether or not subsequent incorporation of the nucleic acid into the genome of the transformed cell occurs.

The term “plant cell” is meant to encompass any cell derived from the plant, including undifferentiated tissues such as callus and suspension cultures, as well as plant seed, pollen or plant embryos. Plant tissues suitable for transformation include leaf tissue, root tissue, meristems, protoplasts, hypocotyls, cotyledons, scutellum, shoot apex, root, immature embryo, callus, somatic embryos, embryogenic structures, pollen and anther.

The term “targeted”, when related to methods for in vivo cell wall polysaccharide remodelling or modification, is meant to distinguish the present invention from mutation approaches which are inherently random in nature, and also distinguish it from transgenic approaches in which polysaccharide precursor pools are altered. The latter approach allows for concurrently changing the monosaccharide profile of all polymers using the affected building block, while the present invention is targeted in the sense that particular cell wall polymers can be modified specifically. In addition, the term “targeted” indicates that side effects on the composition of the targeted polysaccharide, e.g. reducing the amount of fibrillar cellulose or lignin of the wall, are regarded as random modifications. The remainder of the wall following a reduction in any major component will often lead to simple replenishment with other wall building blocks.

The term “complex cell wall polysaccharide” refers to the classes of polymers from vascular plants to which the present invention pertains. Intracellular complex polysaccharides, starch and fructans, are not included. Neither are specialised seed storage polysaccharides exemplified by mannans, galactomannan and storage xyloglucan deposited in secondary walls of seeds. The simple, crystalline fibrils of cellulose does not qualify as “complex” in the present context. Covered by the definition are for example the broad classes of hemicellulose and pectin as defined below.

The term “hemicellulose” or “hemicellulosic polysaccharide” refers to structural xyloglucan (as opposed to storage xyloglucan), xylans, arabinoxylans and various non-cellulosic beta-glucans.

The term “pectin” or “pectic polysaccharide” refers to the group of polysaccharides described above and commonly known as pectins, i.e. polysaccharide materials found in plant cell walls in the form of a mixture of homogalacturonan, xylogalacturonan, rhamnogalacturonan and arabinogalactan polymers.

The term “smooth region” refers to the primarily straight chained, unbranched regions of pectin comprising homogalacturonan, which contain galacturonic acid units which may be esterified to varying degrees, typically with O-acetyl and O-methyl groups. The smooth regions may further contain stretches of xylogalacturonan and rhamnogalacturonan II.

The term “hairy region” refers to the branched chain regions of pectin comprising rhamnogalacturonan I, whose backbone is made up of GalA and Rha and its various side chains containing arabinose and galactose as the major constituents.

The term “polysaccharide-modifying enzyme” refers to any enzyme which is capable of modifying the structure of complex cell wall polysaccharides or any part thereof. A polysaccharide structure is regarded new if it displays a new structure as defined above. In as much as non-sugar decorations are disregarded in the definition of new polysaccharides, enzymes responsible for transfer of these moities are not considered polysaccharide modifying enzymes (see also the definition of “auxiliary enzyme” below). Glycosyltransferases involved in the biosynthesis of the polysaccharide back-bones are not considered herein as a polysaccharide-modifying enzyme either. Manipulating the expression of the synthases may indeed cause major changes to the polysaccharide structures, but, compared to the present invention, this is regarded as a fundamentally different approach to obtaining new polysaccharides, a technique that does not rely on modifications. Yet to be identified genes with roles in single sugar residue decorations of the polysaccharides may be of relevance to the present invention (see definition of “decorative enzymes” below). Polysaccharide modifying enzymes generally belong to the category of enzymes involved in the degradation or turn-over of polysaccharides. Non-limiting examples of these enzymes include endo-rhamnogalacturonan hydrolases, endo-rhamnogalacturonan lyases, endo-polygalacturonases, endo-pectate lyases, endo-pectin lyases, endo-galactanases, endo-arabinanases, endo-xyloglucanases, endo-glucanases, xylanases, arabinoxylanases, xylogalacturonases, arabinofuranosidases, galactosidases, fucosidases, exo-galacturonases and xylosidases. Specific accessions of particular polysaccharide-modifying enzymes of interest include: Aspergillus aculeatus endo-galactanase (AC-number L34599) or an isoform or ortholog thereof, e.g. the ortholog AJ012316 from Aspergillus tubigensis; rhamnogalacturonan lyase (AC-number L35500) or an isoform or ortholog thereof; and Aspergillus aculeatus endo-1,5-alpha-arabinanase (SEQ ID # 1 from WO 94/20611) or an isoform or ortholog thereof, e.g. the ortholog L23430 from Aspergillus niger.

The term “decorative enzyme” refers to an enzyme, such as a transferase, which adds single monosaccharide side chains or non-sugar substituents, typically feroyl, acetyl or methyl esters, to the pectic backbone or side chains. Decorative enzymes are thus set apart from synthase complexes which are also transferases and which undertake polymerisation of the pectic backbone (homogalacturonan and the alternating rhamnogalacturonan backbone) as well as long side chains (most abundantly galactans and arabinans).

The term “auxiliary enzyme” refers to an enzyme which removes a pectin decoration as understood from the definition of a decorative enzyme above. Decoration often renders pectin inaccessible to polysaccharide-modifying enzymes, and hence, co-expression of the latter with a matching auxiliary enzyme enables or optimises interaction between the primary enzyme and its pectic substrate. Examples of auxiliary enzymes include esterases, e.g. a methyl esterase or an acetyl esterase, both of which may be specific to homogalacturonan or rhamnogalacturonan, as well as glycosidases that remove single monosaccharides from polymers, e.g. arabinofuranosidases, galactosidases, xylosidases or fucosidases.

“Molecular farming” refers to the practice of using plants as production vehicles for the production of a particular molecule as opposed to production of vegetables, fruits or fractions thereof (juice or flour for example). Classical examples of plant production for the recovery of well-defined molecules are vegetable oil, sugar and starch. Transgenic technology dramatically increases the range of molecules that can be farmed in plants, and the term is used here with particular reference to these wider perspectives.

The term “ortholog” is used to denote the following relationship between two (enzyme encoding) genes from different organisms: The genes are regarded as being orthologs if sequence similarity indicates evolutionary relatedness (however marginal), their gene products catalyse the same reaction, and they serve essentially similar or overlapping physiological purposes in the two organisms.

The term “isoform” is used to denote the following relationship between two genes from the same organism: The genes encode isoforms of the same gene product if sequence similarity indicates evolutionary relatedness and the encoded enzymes catalyse similar reactions. Isoforms may, or may not, serve different physiological functions in the organism.

The terms “tissue specific” and “organ specific” are used with regard to promoters in such a way that also assimilate inducible promoters are included. Assimilate inducible promoters often direct expression in sink organs solely by virtue of their assimilate, e.g. sucrose, inducibility. For all practical purposes, these are considered storage organ specific in the present context.

Complex cell wall polysaccharides are deposited in the cell wall, i.e. outside the protoplast, while the main steps of biosynthesis takes place in the Golgi apparatus. A polysaccharide-modifying enzyme will therefore come into contact with and act upon its substrate in either of these locations, and in transit from Golgi to the apoplast.

In one aspect, the invention relates to ectopic expression of a plant gene, or heterologous expression of a microbial gene in general without regard to any modification required to ensure proper subcellular localisation. Signal sequences of apoplastic enzymes encoded by plant genes generally function across plant species, also when used in chimeric constructs directing expression to organs where the gene product does not normally accumulate. Even secreted fungal enzymes are trafficked to the apoplast when expressed in plant cells. Those skilled in the art will be familiar with available techniques for engineering a gene where secretion to the apoplast does not function using the native gene, i.e. by replacing a non-functional signal sequence with, typically, a plant or fungal sequence which is known to function, or providing the gene with said sequence without replacement.

Using plant signal sequences ensuring localisation of the transgene product in the Golgi, it is contemplated that persons skilled in the art will be capable of engineering the transgene with appropriate sequences to accomplish modification of cell wall polysaccharides including pectins at their site of synthesis. The below Example 8 describes a particular embodiment of the invention in which advantage is taken of the fact that signal sequences often operate accurately across phylogenetically large distances in that a portion of a rat gene is used to ensure Golgi targeting.

Another aspect of the invention relates to co-expression of a gene encoding an enzyme which removes a particular kind of pectin decoration so as to facilitate access to the substrate of an enzyme which catalyses the desired remodelling of the polysaccharide. This embodiment of the invention is referred to as the use of an auxiliary enzyme. Example 9 hereinbelow illustrates the engineering and transformation involved.

Another aspect of co-expression of enzymes, typically in pairs (unless either enzyme requires an auxiliary enzyme for proper activity), pertains to self-processing plant material (e.g. self-processing tubers in the case of potatoes). This embodiment involves the enzymatic excision of a pre-selected part or a fragment of the targeted cell wall polysaccharide, e.g. pectin, which part or fragment may or may not have been subject to remodelling (in the latter case, the technology may not involve co-expression). Excision of the pre-selected part or fragment occurring post-harvest can be exercised by an enzyme which is stored in the plant cells separately from the substrate, i.e. this enzyme does not catalyse any changes in cell wall polysaccharide structure in the growing plant. Non-limiting examples of possible locations for the enzyme affording the excision include the vacuole, endoplasmic reticulum, cytoplasm and plastids. The location is chosen based upon stability in the plant and possible toxicity to the plant of the gene product in question. The enzyme may, however, also be stored directly in the apoplast if interaction with the substrate (apart for binding) can be avoided either by utilising an enzyme which is substantially inactive at ambient temperatures, or substantially inactive in the pH range found in the plant apoplast (pH 3-7.5) so that the enzyme can be activated by an appropriate change of conditions when desired. Activating treatments include (but are not limited to) heat, addition of salts, addition of organic compounds, physical treatment (pH changes), addition of proteases (e.g. proteases cutting away inhibiting parts of the modifying enzyme), additions of proteins (e.g. another essential subunit of the modifying enzyme).

To summarise, the targeted polysaccharide is remodelled in vivo if desired. The plant is treated post-harvest, e.g. by introducing an incubation step of macerated plant material under conditions dictated by the requirements of the enzyme catalysing the excision, and the stored enzyme is thus allowed to act upon its substrate. The released fragments are then recovered in a state suitable for purification. This technology is superior to the use of technical enzymes in two respects: Technical enzymes are rarely pure enough to act solely by excising a desired part or fragment of a polysaccharide without degrading the product due to the presence of side activities. Many plant organs, storage organs such as potato tubers, provide a very low background of endogenous polysaccharide-modifying enzymes. Additionally, the enzyme affording the excision has excellent access to its substrate compared to a technical enzyme added from the outside, dependent as it is on effective homogenisation of the plant tissue, which is not the case when the enzyme is already present together with its substrate or in a compartment closely connected to the substrate.

The following examples 3 and 10 illustrate retaining of a cell wall polysaccharide-modifying enzyme in the endoplasmic reticulum and targeting of such an enzyme to the vacuole. The below Example 12 provides an example of a particular strategy for designing self-processing plant materials including potato tubers releasing galactanase modified hairy regions, and this example also demonstrates accumulation of a gene product in the cytoplasm.

Applications of the technology presented so far include molecular farming of tailored oligo- and polysaccharides. Use of the technology in any area may in addition lead to valorisation of by-products from other industries. Pulp resulting from beet sugar production and pulp resulting from starch production are non-limiting examples of by-products rich in cell wall polysaccharides including pectin of low value in its native state, but whose value can be improved by means of the present invention.

Medical applications include the provisions of pharmaceutical compounds as well as medical materials such as wound dressings, materials for transplants or implants, biocompatible surgical adhesives, immunomodulating compounds and blood clotting modulators etc. Use of the technology presented herein is not limited to molecular farming of the end-products, but is important already in the discovery phase. Plants producing tailored polysaccharides, such as pectins, can be used for the systematic generation of libraries of polymers and oligomers covering the structure activity space for some medical applications, i.e. to delineate the structural variability allowed while at the same time preserving the functional properties for the application under consideration. It is known per se in the art how to apply chemometric multivariate analysis, QSAR (quantitative structure activity relationships) and QSPR (quantitative structure property relationships) on data from evaluations of such libraries.

For purposes of the present invention, remodelling, i.e. enzymatic modification of the complex cell wall polysaccharides, of any plant belonging to the vascular plants is contemplated, including both monocots and dicots among the angiosperms. With regard to e.g. tailoring of pectin the dicots and non-commelinoid monocots may be particularly important. Conversely, remodelling of hemicellulosic polymers is of significant relevance in some species of the latter category, not least forage grasses and the cereals. Additional non-limiting examples of commercially important plants which may be modified in accordance with the invention include: soybean, tobacco, parsley, carrots, cauliflower, cabbage, broccoli, potato, sweet potato, bean, pea, chicory, lettuce, beet, turnip, radish, spinach, onion, garlic, pepper, celery, willow, poplar, squash, pumpkin, zucchini, cucumber, apple, pear, melon, plum, cherry, peach, nectarine, apricot, strawberry, grape, raspberry, blackberry, pineapple, avocado, papaya, mango, banana, citrus species, Arabidopsis, duck weed, and tomato. Among the non-vascular plants, the algae and the sea-weeds are important.

In specific embodiments the invention is directed to the use of enzymes that modify the hairy regions of pectin, e.g. by cleaving the hairy regions from the smooth regions, and/or by removing or shortening individual side chains of the hairy regions. For this reason, nucleotide sequences that result in expression of such enzymes, so as to result in transgenic plants in which pectin is modified in vivo in the hairy regions, are important. In the particular embodiment of the invention referred to as self-processing plant material”, enzymes which render parts of the polymer more soluble,. or release it from the wall matrix, are important. The focus here is thus on enzymes which cleave the polymeric backbone of the polysaccharide in question.

As persons skilled in the art will be aware, the cell wall polysaccharides in questions are quantitatively and functionally important constituents of plant cell walls. This being the case, it must be assumed that nature sets certain limits as to the extent of modifications that are possible. It is therefore clear that the modifications performed in the context of the present invention should not be so drastic so as to result in a non-viable plant. Some developmental changes resulting from growth impairing cell wall changes may be exploited to obtain localised cell ablation as a route to male sterility and other desirable traits already mentioned. On the other hand, as indicated above, the present inventors have surprisingly found that significant modifications of the monosaccharide profile and the overall linkage pattern of cell wall polysaccharides are possible, while still maintaining viable plants. Based on these surprising findings, it is contemplated that using the teachings herein, a wide variety of modified cell wall polysaccharides may be produced in vivo without sacrificing plant viability.

Ways of Modifying Complex Cell Wall Polysaccharides

In accordance with the invention, complex cell wall polysaccharides may be modified either in planta or following harvest of the plant material in question in several different ways. These will be briefly summarised in the following:

(i) Manipulation of biosynthesis either by modulation of plant genes involved directly in biosynthesis of complex cell wall polysaccharides e.g. the transfer and polymerisation steps, by manipulation of pool sizes and transport of substrate molecules or by interference with polymer biosynthesis by expression of polysaccharide-modifying enzymes which, following translation and entry into the secretion pathway, are retained in the Golgi apparatus, hence forcing turn-over of the pectic or hemicellulosic carbohydrates at their place of synthesis.

(ii) Post-deposition modification by expression of appropriate polysaccharide-modifying enzymes equipped with signals which ensure post-translational transport directly to the apoplastic space and hence the cell wall, resulting in direct interaction with the enzymatic substrate in question after its apposition to or intussusception into the cell wall.

(iii) Post-harvest release of one or more polysaccharide-modifying enzymes which have been produced in planta followed by post-translational accumulation in an appropriate cellular organelle or compartmentalised indirectly through the selection of enzymes that are active only under the post harvest conditions, and not in the living plant.

Organ/tissue Specific Expression of Cell Wall Polysaccharide-modifying Enzymes

It is contemplated that strategies which target expression of the polysaccharide-modifying enzymes of the invention and thus production of the modified cell wall polysaccharides to only certain parts of a given plant will make possible an even greater range of in vivo cell wall polysaccharide modifications. For example, it is contemplated that in potatoes, it will be advantageous to express the polysaccharide-modifying enzymes in the tubers only, whereas other parts of the plant will maintain their native polysaccharide structures.

Such targeted expression of polysaccharide-modifying enzymes according to the present invention may suitably be performed using tissue specific regulatory sequences. Many such tissue specific sequences are known in the art.

U.S. Pat. No. 5,723,757 e.g. discloses the use of 5′ transcriptional regulatory regions of plant genes which ensure “sink tissue” specific expression of a desired DNA sequence. Such transcriptional regulatory regions allow expression of a desired DNA sequence to specifically take place in “sink organs”, i.e. photosynthetically inactive parts of a plant such as roots, grains, fruits or tubers. As a result, gene products of interest can be either expressed or inhibited in certain tissues. The regulatory regions are in particular derived from genes coding for patatin proteins belonging to class I patatin genes.

U.S. Pat. No. 5,436,393 discloses the DNA sequence of an expression cassette comprising potato tuber specific regulatory regions from the patatin gene from potato, in particular the B33 promoter sequence of a patatin gene. Transformation of potato cells using an expression cassette makes possible the production of transgenic potato plants in which a DNA sequence of heterologous origin, fused to the B33 promoter sequence, can be expressed in a tuber specific manner.

Other known promoters directing expression to tubers include the putative metallocarboxypeptidase inhibitor from potato (accession number U30388), the potato lipoxygenase (accession number X95513) and the cathepsin D inhibitor (accession number X74985). Persons skilled in the art will be able to excise other promoter regions having a desired specificity and prepare chimeric gene constructs with the desired polysaccharide-modifying enzyme. As at least some tuber specific promoters owe their organ specificity to their sucrose inducibility, it is not surprising that these promoters often will be operational in organs of other species where sucrose accumulates, for example beet roots (the activity of the GBSS promoter in tobacco leaves is illustrated in Example 10 herein). Conversely, it will not be surprising to those skilled in the art that sucrose inducible promoters can be identified from other species and be found to be not only storage organ specific in the originating species but also to confer tuber specificity in potato. Promoters that ensure high expression e.g. in tubers of potato include the GBSS and the B33 promoter.

Modifying Biosynthesis of Plant Cell Wall Polysaccharides

Opportunities for remodelling of pectic or hemicellulosic polysaccharides will be available using enzymes which act at the different levels of synthesis: (i) maintenance of the pool of nucleotide sugars, (ii) polymerisation of a particular backbone, and (iii) decoration of these backbones with various substituents (glycosyl residues, methyl and acetyl groups).

Interference with biosynthesis can, in accordance with the invention, be accomplished by targeting a polysaccharide-modifying enzyme to the Golgi apparatus to interfere with biosynthesis. The enzyme can be engineered so that it becomes membrane anchored, and thus retained in the Golgi, or it can be targeted to the Golgi as a soluble enzyme which eventually will be secreted along with the cell wall polysaccharides into the apoplast. It was a surprising finding that an enzyme which is anchored to the Golgi membrane successfully interacts with a biosynthetic complex which itself is bound to the membrane. Hence both the membrane anchored and the soluble variations are of interest, with the former expected to be most specific. This embodiment also affords a possible solution to the problem arising from possible toxicity to plants of some secreted polysaccharide hydrolases.

Several lines of evidence support the view that oligogalacturonides released through enzymatic degradation of the plant cell wall by elicitor enzymes can trigger specific responses in the plant cell (Moloshok et al., 1992). Therefore, it is likely that one or more of the enzymes which, in accordance with the invention, are potential candidates for in vivo modification of the plant cell will release carbohydrate oligomers that can initiate a response similar to the one observed for oligogalacturonides.

It has been found that the N-terminal 77 amino acids from tobacco N-acetylglucosaminyltransferase I are sufficient to retain a reporter protein in the Golgi apparatus of Nicotiana benthamiana cells. Essl et al. (1999) and Dhugga et al. (1997) cloned polypeptides with an implied role in polysaccharide synthesis, and these polypeptides are Golgi associated without being integral proteins (Pisum sativum reversibly glycosylatable polypeptide (RGP1) U31565). From Arabidopsis an acetylglucosaminyltransferase AJ243198, and a xylosyltransferase, AJ272121, have been identified. Following the complete sequencing of the Arabidopis genome, those skilled in the art will know how to search the genome for additional Golgi targeted gene products. As additional soluble and integral plant Golgi proteins are characterised, sequences will become available for engineering polysaccharide-modifying enzymes for Golgi targeting. At present, one may take advantage of the fact that at least some Golgi targeting sequences are functional between very unrelated species. In one specific embodiment of the invention, a sequence from a rat gene can used such as it is demonstrated in Example 8 herein.

Post Deposition Modification of Cell Wall Polysaccharides

The below Example 5 illustrates the use of a fungal galactanase for the targeted change of the monosaccharide profile of a pre-selected part or fragment of a cell wall polysaccharide as exemplified by the pectin hairy region. Galactan side chains are shortened to an extent where the side chains are no longer substrates for the enzyme. The plant compensates by increasing the relative and absolute content of uronic acids and by increasing the degree of acetylation.

In the present case the galactans contained few or no decorations with short monosaccharide side-chains (most typically arabinosyl decorations), so that co-expression of an auxiliary enzyme was unnecessary. Where necessary, a particular embodiment of the invention employs double constructs charged with the remodelling enzyme of interest, plus the auxiliary enzyme which co-acts during the processing, see Examples 4 and 9 below.

Examples 2 and 7 herein illustrate the expression of a rhamnogalacturonan lyase, i.e. an enzyme which nicks the pectin hairy region backbone and it was demonstrated that this enzyme is capable of cleaving the pectin polymer irrespective of its decoration with acetyl and methyl esters. Where this is not the case, an auxiliary enzyme, typically a rhamnogalacturonan or homogalacturonan acetyl esterase or a methyl esterase, is co-expressed with the primary remodelling enzyme to afford back-bone nicking.

Post-harvest Modification and Self-processing Plant Material

Self-processing plant material, e.g. tubers, is designed by storing one or more enzymes in the plant tissue as already explained so as to prevent their interaction with its/their substrate in vivo while it/they will excise a desired fraction post harvest. This expression may be coordinated with expression of additional enzyme(s) catalysing the remodelling of the target cell wall polysaccharide during growth. As mentioned above, sequestering of the processing enzyme(s) during growth of the plant may e.g. be in the vacuole, endoplasmic reticulum, cytoplasm and/or plastids, or it may be through chemical sequestering. The internally stored enzyme may e.g. be targeted to the endoplasmic reticulum using the KDEL retention signal or to the vacuole using a signal sequence derived from patatin or sporamin.

Upon being subjected to a treatment post-harvest, typically a physical treatment in the form of homogenisation or maceration, the internally stored enzyme is brought into contact with the cell wall polysaccharides to catalyse a post-harvest modification of the cell wall material. One important and interesting embodiment of post-harvest modification is the release of the in vivo modified polymers from pulp material. Using tailored pectin hairy regions as an example, these are released by the action of an enzyme which cleaves the smooth regions of the pectin. Example 12 below illustrates targeting of plant or fungal endo-polygalacturonases to the cytoplasm, to the vacuole and retained in the endoplasmic reticulum (ER). The site inside the cell is selected based on stability and attained accumulation in each site of the enzyme as well as on possible toxicity of the enzyme to the plant when stored in different locations. Conditions for maceration and incubation of the plant material for self-processing are chosen according to properties of the internally stored enzyme, the occurrence of interfering endogenous enzymes in the plant tissue and possibly of the stability of the polymers or oligomers to be recovered. It will be realised from the foregoing that either enzyme may require an auxiliary enzyme, and hence, that ternary or higher order vector constructs can be developed to thus extend this embodiment of the invention.

Enzymes with the desired property of being able to release pectic polysaccharides from the cell wall polysaccharide mesh comprise in particular those enzymes which (optionally assisted by an auxiliary enzyme) cleave a polysaccharide backbone. Pectin and pectate hydrolases and lyases, rhamnogalacturonan hydrolases and lyases are known enzyme categories with this property. Enzymes active against xylogalacturonan will be similarly useful and should be regarded as exemplifying new activities against specialised structural features of the polysaccharide backbone. Enzymes capable of removing pectic side chains are in the present context viewed primarily as remodelling enzymes. However, it is evident that there may well be examples where side chain removal will solubilise particular oligosaccharides.

It is evident that the technology allowing internal storage of the enzymes is useful for the molecular farming of the enzymes for their own sake. Likewise, it is evident that the technology is useful for the manufacture of functional feed. By “functional feed” is meant-fodder for livestock and poultry where one or more intercellularly accumulated polysaccharide-modifying enzymes enhance the digestibility of the plant material itself as well as that of other feed ingredients with which it has been mixed. Example 10 demonstrates accumulation of an arabinanase in the ER of tobacco leaves.

Food Industrial Applications of Modified Pectin or Fragments Thereof

Pectin is an important food additive and ingredient and it is known to be an important factor in determining the texture of plant material. Modification of its structure, especially at the regions where the molecule is heavily branched (hairy regions) will alter both the physical properties of the polymers and the properties of the cell wall material. This technology allows the ratio of neutral sugar side-chains to homogalacturonan to be manipulated. The functional properties of the pectic phase which correlate with this ratio, and which are of major interest in the food industry, are gelling, gel stability and water-binding efficiency, with the latter being favoured in polymers with larger proportions of hairy regions, and the first two in polymers with a high content of homoglacturonan.

Potato pectins have not found any use in food applications due to their high neutral sugar content and the presence of acetyl groups preventing gelation. From a historical point of view, the use of high quality lemon and lime pectins has usually been directed to applications in jellies, jam etc. (high methyl-ester pectins) on the one hand and in low sugar products (low methyl-ester pectins such as calcium pectates) on the other hand. In the last decade, however, the field of application has broadened and now includes use as an emulsifier and stabiliser and as a coating for products prior to frying. New applications for pectin derived molecules include use as a drinkable dietary fibre. Here it is a problem that pectin gives a rather high viscosity. It has, however, been shown that when degrading the homogalacturonan part of pectins, the rhamnogalacturonan part (Modified Hairy Region—MHR) maintains the same fibre effect while at much lower viscosity (EP 0868854-A2). Similar MHR can be obtained according to the invention from tailored pectins produced by potatoes. Keywords here are low cost and large volume of raw material, both characteristics of the potato pulp raw material. Ideally, a potato can be produced that incorporates PG-activity activated during the starch processing, giving the modified hairy regions directly in the potato juice, i.e. self-processing tubers are of relevance here. This will give both easier starch processing and low cost modified hairy regions. Easier starch processing may result from promoted extractability or from “less packaging” tuber cells, i.e. cells where a direct wall thinning has taken place as an intended tailoring of the wall or as a side effect of pectin remodelling. Apart from the starch extraction which is relevant only to starch storing organs of course, neither the problems associated with low quality pectin from some (abundant) sources, nor the solutions afforded by the technology presented here, are potato specific. The technology is relevant for other crop plants as well, e.g. for sugar beet.

More specialised food applications require control over water binding capacity and over viscosity and “mouth feel” of the polymeric solutions. Rhamnogalacturonans, native or tailored, may find uses as low viscosity products like milk shakes, drinking yoghurt, etc.

Pectin engineering would furthermore be useful for the juice, cider and wine industry and would hence be relevant for grapes, apples and other fruits. Juice and must are often subjected to filtering to remove hydrocolloids and suspended oligo- and polysaccharides so as to clarify the juice and prevent the occurrence of haze or precipitates in later processing steps. Filtering may be facilitated by tailoring pectin so as to increase solubility, or conversely to increase precipitability. The present invention provides new opportunities for gaining control over those juice and must characteristics which rely on soluble and suspended pectic polymers.

Finally, since pectins mediate the cell wall porosity (Baron-Epel et al, 1988), tailored pectins may influence calcium firming and similar processes of relevance to canning. The invention therefore provides a method for improving food industrial processability of plant material in general, e.g. with respect to canning, calcium firming, filtering, etc., by using plant material with modified pectin as described herein.

Medical Applications of Modified Plant Cell Wall Polysaccharides

In addition to the various possibilities discussed above for modifying pectic polysaccharides for various industrial applications, it is also contemplated that modified cell wall polysaccharides including modified pectin substances will be useful for a variety of applications in the pharmaceutical and medical fields. For example, the present invention makes it possible to modify the structure of a cell wall polysaccharide in vivo to produce a polysaccharide modified to have specific properties with respect to e.g. immunomodulation. Further, it is contemplated that modified cell wall polysaccharides developed and produced in accordance with the present invention may be suitable for general use as antiinflammatory compounds for any type of inflammatory reaction, whatever the cause, including inflammation resulting from physical damage, chemical agents, and biological agents such as bacteria, fungi, viruses and other microorganisms.

Use of the tailored complex cell wall polysaccharides falls into two basic areas, pharmaceuticals and medical materials. Materials currently used for the manufacture of wound dressings for chronic, non-healing wounds, for wounds caused by psoriasis (which has an auto-immune component to the disorder), for wounds involving gangrene and for ostomy products rely almost entirely on alginates and modified celluloses selected for their physical properties (which they generally share with many pectic and hemicellulosic polymers). For wound dressings aimed at treating various types of chronic wounds, physicochemical properties are important. These include gelling ability and gel strength, water binding capacity, charge density and hydrophilicity. These properties are well studied in relation to food industrial uses of plant polysaccharides. Modified cell wall polysaccharides will be able to act as a water buffering system where the dressing either removes water produced by the wound, or supplies it to dry gangrene so that the cellular immune system gains access to the surface. The biological activities of pectin, however, are unique to pectins. Desirable bioactivities include the potential of the pectins to act as a reservoir for cellular growth factors and hence stimulate cell growth in the chronic wound.

Another interesting perspective is to use a pectin membrane as a scaffold for regrowing skin from cultured skin cells to be used as transplants.

Use as drugs to inhibit autoimmune processes or to prevent or treat the effects of autoimmune processes is contemplated to be applicable to a broad range of disorders. Certain types of ulcers are believed to involve an autoimmune aspect, and also in these cases pectic structures likely to be rhamnogalacturonans have been found to be active (Sakurai et al. 1998). Further non-limiting examples of such autoimmune-associated conditions include: Autoimmune hepatitis, Primary biliary cirrhosis, Primary sclerosing cholangitis, Autoimmune hemolytic anemias, Grave's disease, Myasthenia gravis, Type 1 Diabetes Mellitus, Inflammatory myopathies, Multiple sclerosis, Hashimoto's thyreoiditis, Autoimmune adrenalitis, Crohn's Disease, Ulcerative Colitis, Glomerulonephritis, Progressive Systemic Sclerosis (Scleroderma), Sjögren's Disease, Lupus Erythematosus, Primary vasculitis, Rheumatoid Arthritis, Juvenile Arthritis, Mixed Connective Tissue Disease, Psoriasis, Pemfigus, Pemfigoid, Dermatitis Herpetiformis, etc.

Another contemplated use for modified pectins and other polysaccharides is in combating some types of cancer. There is evidence to suggest, for example, that modified citrus pectin (MCP) is able to combat various types of cancer, including melanoma (skin cancer) and prostate cancer. This effect is believed to be associated with a cytostatic or “antiadhesive” effect provided by the MCP in the early stages of metastasis (Ralph W. Moss, http://ralphmoss.com/mcp.html). By use of the method outlined herein for identifying and producing modified pectic polysaccharides, it is believed that novel modified cell wall polysaccharide substances including modified pectins suitable for use as anti-cancer agents can be developed and commercially produced e.g. by means of molecular farming techniques.

Applications in the medical field are not restricted to the production of pharmaceuticals and medical materials but are also relevant for the precedent discovery phase, i.e. as a tool for generating libraries of cell wall polysaccharide oligo- and polysaccharides and for determining the structural requirements for a desired pharmaceutical activity of such oligo- and polysaccharides. E.g. pectic polymers are synthesised as repeating structures which in turn consist of other, smaller repeating structures. While the remodelling technology described herein does not change the fact that pectic polymers remain less than fully characterised, it is possible to produce populations of molecules in which the difference between molecules is rather precisely known. For example, rhamnogalacturonan from tubers expressing endo-galactanase described in Example 5 differs from the corresponding wild-type polymers by the length of the galactan hairs, degree of acetylation and uronic acid content. Hence, it can be determined with certainty whether these features are essential for an observed bioactivity.

Using pectin as an example, a library in which the differences between entries are known (even though the precise structures are not) can be generated using the following steps:

1) Tailored pectins are recovered from a range of transgenic plants each producing polysaccharides modified in a well-defined way.

2) Samples from step 1 are subdivided and treated post-harvest with enzymes in vitro.

3) Samples from step 2 are subdivided and modified by chemical treatments, saponification and partial hydrolyses for example and derivatisation in vitro; permethylation, acetylation, epoxidation and amidation are non-limiting examples of the latter. Types of derivatisations and methods are known per se from the food ingredient industry, the textile industry and from organic synthesis and spectroscopy of carbohydrates.

Step 2 and 3 may, if desired, be performed in the opposite order so that the chemical modification serves the purpose of protecting or exposing some parts of the molecule to enzymatic reactions.

The library entries are then subjected to screening for medical activity using screening procedures known per se in the art. As the structure-function relationship of particular compounds becomes evident, this will provide information useful for deciding whether to transfer some of the enzymatic treatments used post-harvest (step 2) into the plant (where compensation by the plant allows for it), and thus develop transformed plants for the molecular farming of a starting material which is tailored as much as possible in the plant by means of in vivo processing of pectin.

The invention will be further illustrated by means of the following non-limiting examples and the drawings wherein:

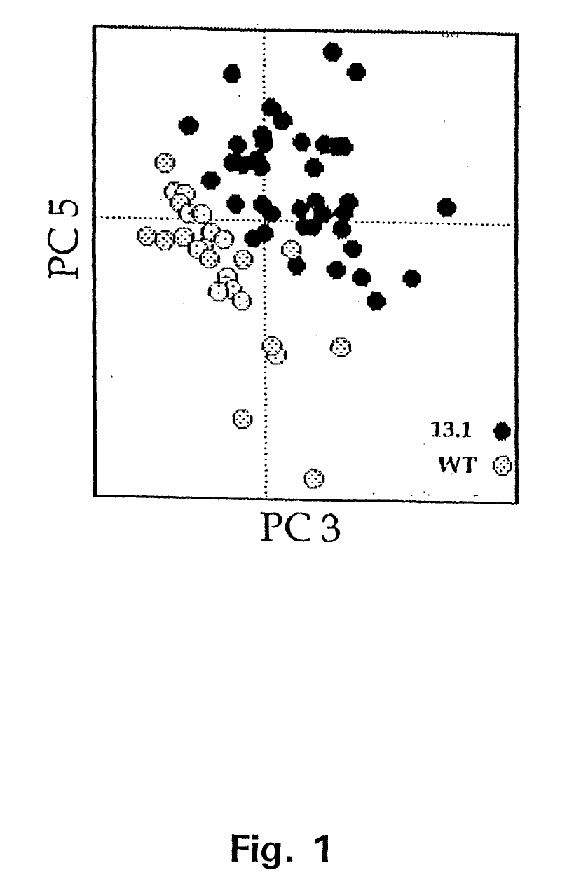

FIG. 1 illustrates the use of exploratory principal component analysis (PCA) to discriminate two transgenic potato plants, T_{11.1} and T_{13.1}, expressing Aspergillus aculeatus endo-galactanase from wild type, and it shows the comparison between T_{13.1} and wild type, using the third and fifth Principal Component (PC) score;

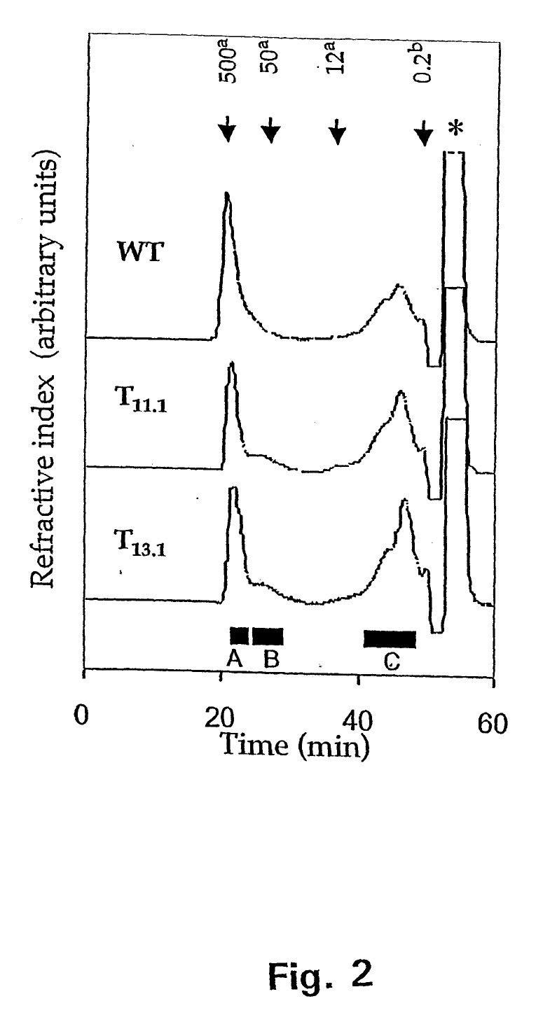

FIG. 2 shows size exclusion chromatographic analysis (detection by refractive index) (arbitrary units) of rhamnogalacturonan I (RGI) specifically extracted from cell walls of wild type potato tubers (WT) and transgenic potato plants, T_{11.1} and T_{13.1}, respectively by treatment with a combination of fungal EPG and PME. This EPG/PME treatment released nearly twice as much uronic acid (UA) from the cell walls of transformed tubers compared to wild type. The RGI extracted by EPG/PME from wild type cell walls contained two major fractions as indicated by UA content and refractive index detection: fraction A (molecular weight>500 kDa) and fraction C (molecular weight 0.2-8 kDa). EPG/PME extracts from T_{11.1} and T_{13.1} have a different profile from the wild type containing less of fraction A, substantially more of fraction C and in addition, fragments of ˜120 kDa (fraction B), not present in wild type extracts. The asterisk indicates a large peak due to the presence of sample buffer salts devoid of pectic material; and

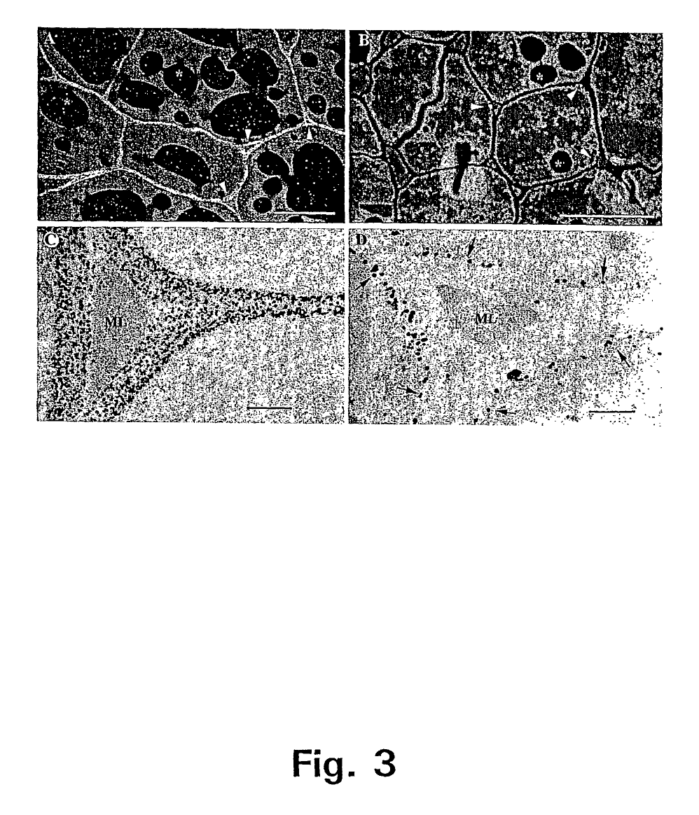

FIG. 3 shows sections of wild type (A, C) and endo-galactanase-expressing (T_{13.1}) (B, D) potato tubers gold labelled with monoclonal antibody LM5, silver enhanced and viewed by reflection confocal scanning microscopy (A, B) and transmission electron microscopy (C, D). The walls of wild type parenchymal cells are strongly labelled (white in A, black particles in C), whereas in T_{13.1} tubers, the labelling density is greatly reduced and localised only to some cell corners (arrow heads in B) close to the plasma membrane (arrows in D). Asterisks represent spaces once occupied by starch granules. ML indicates the expanded middle lamella of these filled corners. Scale bars: A and B 100 mm, C and D 2 mm.

### EXAMPLES

Materials and Methods

Reagents and Enzymes

All enzymes used for general molecular biology methods were purchased from Boehringer, Denmark, unless otherwise stated. Chemicals and reagents for cell wall characterisations were purchased from Sigma. The porcine pancreatic alpha-amylase was purchased from Merck (Darmstadt, Germany), the pullulanase from Bacillus acidopullulyticus and endo-polygalacturonase from Aspergillus niger were purchased from Megazyme International (Bray, Ireland) and the purified recombinant pectin methylesterase (PME) from Aspergillus aculeatus was a gift from Dr. Kirk Schnorr (Novo Nordisk A/S, Bagsvaerd, Denmark).

Transformation of Plants

Transformation of Nicotiana tabacum (L.) cv. Xanthi. leaf discs was performed essentially as described by Horsch et al., 1985. Internodes, from in vitro plants of both transformants and wild type plants, were transferred to the greenhouse to generate mature plants. Leaves were harvested and used for analysis. Potatoes, cv Posmo and Karnico were used for Agrobacterium tumefaciens mediated transformation (Visser, 1991). In vitro shoots of transgenic and control clones were transferred to the greenhouse to generate mature plants.

DNA Analysis

DNA was extracted from leaves ground in liquid: N₂ according to the CTAB protocol as described by Rogers and Bendlich (1988). For determination of the presence of the transgene in the transgenic clones, isolated genomic DNA was digested with EcoRI, which cuts twice within the cDNA, and KpnI, which cuts once within the cDNA. The digested DNA was separated by electrophoresis and blotted onto Hybond N membranes (Amersham) under alkaline conditions as described by Sambrook et al. (1989). The membranes were hybridised, as described by Salehuzzaman et al. (1992), with a ³²P-ATP labeled 1 kb KpnI-XbaI cDNA fragment of rhamnogalacturonan-lyase/pYES2.

RNA Analysis

RNA was extracted from 1 tuber of each transgenic line as described by Kuipers et al. (1994). Tissue (5 g) was ground in liquid N₂ and mixed with 5 ml of extraction buffer (50 mM Tris, pH 9.0, 10 mM EDTA, 2% SDS) and 5 ml of phenol. The mixture was centrifuged (10 min at 2,000 g), the supernatant was extracted with 5 ml of phenol/chloroform/isoamyl alcohol (25:24:1), and the mixture was centrifuged again. Nucleic acids were precipitated with 5 ml of isopropanol and centrifuged (10 min at 2000 g); the pellet was dissolved in 1125 μl H₂O. RNA was precipitated overnight at 0° C. with 375 μl of 8 M LiCl and centrifuged (10 min at 13,000 g). The RNA pellet was dissolved in 0.4 ml of H₂O and precipitated with 40 μl of 3 M NaAc and 1 ml of ethanol. Following centrifugation the pellet was washed in 70% ethanol, dried, and resuspended in H₂O. RNA gel blotting and hybridisation were carried out using 40 μg of tuber RNA per sample, as described by Sambrook et al. (1989). The membranes were hybridised with the following ³²P-ATP labelled cDNA probes: a 1 kb KpnI-XbaI fragment of rhamnogalacturonan-lyase/pYES2 and a 2.3 kb EcoRI fragment of a potato 28S ribosomal RNA gene (Landsmann and Uhrig, 1985).

Preparation of Leaf and Tuber Extracts

Freshly harvested leaves were frozen in liquid N₂ and comminuted using a pestle and mortar with 3 ml extraction buffer A (25 mM NaOAc pH 5.0 containing Complete TM, Boehringer Mannheim) per g tissue (fresh weight). The sample was incubated for 10 min. on ice and insoluble material precipitated by centrifugation (18,000×gₘₐₓ) for 10 min., the supernatant was recovered and stored at 20° C. Freshly harvested tubers were cut into small cubes, frozen in liquid N₂ and comminuted in an electric grinder to a fine powder. The powder was extracted with extraction buffer A as described above.

Galactanase Activity Assays

The enzyme activity was determined using a plate assay with a 0.5% upper and 1% lower agarose layer in 50 mM sodium citrate pH 4.5. The upper layer contained a suspension of the substrate, azurine-coupled potato galactan (Megazyme International, Bray, Ireland), at a concentration of 1 mg/ml. Aliquots (20 μl ) of tissue supernatants were added to wells punched in the upper layer only. Plates were incubated at room temperature for 24 hr.

Soluble, quantitative galactanase assay. Tuber extracts of wild type and transformants (T_{11.1} and T_{13.1}) were assayed for endo-galactanase activity using the p-hydroxybenzoic acid hydrazine assay (Lever 1972) with potato galactan (P-GALPOT from Megazyme) at a concentration of 0.1% in 0.1 M NaOAc pH 4.0 as substrate at 40° C.

Arabinanase Activity Assays

The enzyme activity was determined using a plate assay with a 0.5% upper and 1% lower agarose layer in 50 mM sodium citrate pH 5.5. The upper layer contained a suspension of the substrate, azurine-coupled sugar-beet arabinan (Megazyme International, Bray, Ireland), at a concentration of 1 mg/ml. Aliquots (20 μl) of tissue supernatants were added to wells punched in the upper layer only. Plates were incubated at room temperature for 24 hr.

Aspergillus RG-lyase Assay

Frozen potato tissue was ground with a mortar under addition of liquid nitrogen. Approximately 1 gram of ground potato tissue was homogenized using an Ultra-Turrax TP 18-10 (14,000 rpm, Ika Werk, Staufen, Germany) in 2 ml of a 0.25 M sodium phosphate buffer (pH 6.5; 4° C.) containing 0.4 M NaCl. After 1 h of extraction at 4° C. (with periodic shaking), the suspension was centrifuged (10 min, 2,000 g). The supernatant was used as the enzyme extract.

Subsequently, 5 μl of supernatant was added to 245 μl of a 0.25% w/v solution of saponified apple MHR in 0.1 M sodium phosphate buffer pH 5.0. After incubation for 15 min. at 40° C. in a vortex mixer, the enzymes were inactivated by heating for 5 min. at 100° C. Degradation of the saponified apple MHR was determined using HPSEC (BioGel TSK 40XL, 30XL, and 20XL columns in series).

SDS-PAGE and Western Blot

Tuber extracts were subjected to SDS-PAGE and Western Blot analysis according to standard procedures. The blot was probed with a rabbit antibody raised against purified Aspergillus aculeatus galactanase (a gift from Jens-Christian Navarro Poulsen, Centre for Crystallographic Studies, University of Copenhagen).

Fourier Transform Infrared Microspectroscopy

Epidermal peels from 20 leaves from in vitro plants and Vibratome sections (60 μm) from 20 individual freshly harvested wild type and transgenic (11.1, 11.2 and 13.1) tubers were mounted on barium fluoride windows and air dried. The barium fluoride window was supported on the stage of a UMA500 microscope accessory of a Bio-Rad FTS175c FTIR spectrometer equipped with a liquid nitrogen-cooled mercury cadmium telluride detector. Areas of 100 μm² of the cortex and perimedullary regions were selected and spectra obtained. Sixty-four interferograms were collected in transmission mode with 8 cm⁻¹ resolution and co-added to improve the signal-to-noise ratio for each sample. The spectra were baseline-corrected and area-normalised. Exploratory Principal Component Analysis (PCA) of area-normalised spectra in the region 1800 to 800 cm⁻¹ were carried out using WINDISCRIM software (E. K. Kemsley, Institute of Food Research, Norwich, UK).

Analysis of the Pectin in Transgenic Potato Tubers

To determine the fine structure of the pectin in potato plants it is first necessary to isolate the cell wall material (CWM) from the potato tuber. Potato tubers were peeled, diced and frozen under liquid nitrogen. Subsequently, 100 g of tissue was chopped into a powder using a Braun Multimix MR 860 kitchen chopper (2×30 sec, 350 watt). The potato powder was homogenised with 300 ml of mixed-cation buffer (10 mM NaOAC, 3 mM KCl, 2 mM MgCl₂ and 1 mM CaCl₂), containing Triton X 100 (2 mg/ml) at 4° C. for 7×60 s using an Ultra-Turrax T25 (24,000 RPM). A few drops of octanol were also added to minimise frothing during blending. The detergent was removed by washing through a 250 and 36 μm sieve with chilled mixed-cation buffer. This procedure was carried out at 4° C. The washed residue was removed from the sieve and stirred in chilled 50% acetone. The sample was filtered and weighed into a large beaker. Five times the sample weight of a saturated phenol solution was added. After 30 minutes stirring the saturated phenol was removed by suction and the residue was washed with the mixed-cation buffer solution on a grade 3 sintered glass funnel.

The residue was frozen into small pellets in liquid nitrogen and cryomilled for 2×15 seconds in a coffee grinder. Immediately after thawing, the sample was mixed into a paste with mixed-cation buffer. This paste was stirred quickly into a large volume of boiling mixed-cation buffer and boiled for 30 seconds. Immediately following the 30 second boil the mixture was decanted into chilled mixed-cation buffer (10 volumes), resulting in immediate cooling.

Starch removal was effected with ca 1,500 units of alpha-amylase (Boehringer) and 400 units of pullulanase (Megazyme) in mixed-cation buffer with 0.01% w/v NaN₃. The sample was agitated in an orbital shaker at 37° C. overnight. If starch still remained, the enzyme treatment was repeated. The presence of starch was detected using 1% Kl/0.5% I₂ under the light microscope. When starch removal was complete as assessed by Kl staining the cell walls were washed on a 36 μm sieve to remove glucose and salt. The residue was freeze-dried as the cell wall material.

The obtained CWM was fractionated by extraction with different solvents as described by Huisman et al. (1999). For 1 gram of CWM extract twice with 70 ml 0.05 M sodium acetate buffer pH 5.2 for 60 min. at room temperature, three times with 40 ml of 0.05 M sodium acetate buffer pH 5.2 for 45 min. at 70° C., three times with 35 ml 0.05 M sodium acetate buffer pH 5.2 containing 0.05 M EDTA and 0.05 M ammonium oxalate for 45 min. at 70° C. and finally three times with 40 ml 0.05 M sodium hydroxide for 45 min. at 2° C. After each extraction step, the suspension was filtered over a grade 3 sintered glass funnel.

This resulted in five different fractions, Cold Buffer Soluble Solids (CBSS), Hot Buffer Soluble Solids (HBSS), Chelating Agent Soluble Solids (ChSS), Alkali Soluble Solids (ASS) and a hemicellulose rich residue (Res), each containing a different population of cell wall polymers. Before further analysis, all fractions were freeze-dried.

The CWM and the fractions were characterized using different techniques: Neutral sugar composition was determined by subjecting the samples to a 1 M H₂SO₄ hydrolysis (3 h at 100° C.). Next, the released neutral sugars were converted into their alditol acetates and separated on a 2-mm i.d. glass column packed with Chrom WAW 80-100 mesh, coated with 3% OV275 (Chrompack, Middelburg, The Netherlands) in a Carlo Erba Fractovap 2300 gas chromatograph (Milan, Italy) operated at 200° C. and equipped with a flame ionization detector set at 270° C. Inositol was used as the internal standard.

The degree of acetyl and methyl esterification was estimated by HPLC using the procedure desribed by Voragen et al (1986).

Pectinases, e.g. polygalacturonase, pectin methyl esterase, endo-arabinase, endo-galactanase, arabinofuranosidase, galactosidase, pectate lyase, rhamnogalacturonan acetyl esterase, in combination with High Performance Size Exclusion Chromatography (BioGel TSK 40XL, 30XL, and 20XL columns in series) and High Performance Anion Exchange Chromatography using a Dionex (Sunnyvale, Calif., USA) Bio-LC GPM-II quaternary gradient module equipped with a Dionex CarboPac PA-100 column (250×4 mm, 20° C.) were used to further elucidate the structure of the pectin fractions. However, solubility problems hindered these experiments.

The results obtained from structural analysis of wild type potato pectin will be used for comparison with the transgenic potato pectin. It is believed that the solubility problems that occurred during the enzymatic characterization can be overcome by keeping the fractions in solution instead of freeze-drying them. Furthermore, MALDI-TOF MS can be used to obtain additional information by studying the fragments released by pure and well characterized enzymes.

Isolation of Pectic Polysaccharides

Following the procedure of O'Neill et al. (1990), de-starched cell wall material (10 mg) was suspended in 2 ml 50 mM ammonium formate, pH 4.5, containing 0.05% sodium azide. EPG (1 u, 1 unit releases 1 μmol of reducing galacturonate per min) and PME (1 u, 1 unit releases 1 μmol of methanol per min) were added, and the suspension incubated for 16 h at 40° C. The suspension was then filtered through a double layer of nylon (30 μm pore size) to separate the EPG/PME extracts from the remaining wall material which was suspended in 1 ml ice-cold 50 mM sodium carbonate containing 10 mM sodium borohydride. This suspension was incubated for 1 h at 4° C., followed by a 4 h incubation at room temperature. Insoluble material was recovered by filtration through a double layer of nylon. The pH of the filtrate was adjusted to pH 5, EPG (1 u) was added and the mixture was incubated for 4 h at 40° C. to partially depolymerise solubilised pectic material. This digested soluble wall material was termed the carbonate extract.

Both the EPG/PME and carbonate extracts were subjected to size exclusion chromatography using a Superose 12 column (1×30 cm, Amersham Pharmacia Biotech, Uppsala, Sweden). The column was equilibrated in 50 mM ammonium formate, pH 5.0) before the extracts were applied to the column and eluted isocratically with the same buffer at a flow rate of 0.4 ml/min. The eluent was monitored using refractive-index detection (Model 131, Gilson, Middleton, Wis.) and the uronic acid content of the collected fractions (0.8 ml) determined using the m-hydroxybiphenol assay (Blumenkrantz & Asboe-Hansen 1973). Certain fractions were pooled, freeze-dried and their monosaccharide composition determined. The molecular mass of the eluted components was estimated by comparing their retention times to those of dextran standards (Fluka, Buchs, Switzerland) and galacturonic acid.

Monosaccharide Composition Analysis

Seaman hydrolysis (Selvendran et al., 1979) was used to hydrolyse crude or insoluble cell wall residues for monosaccharide composition analysis, whereas solubilised wall fractions were hydrolysed to monosaccharides using 2 M aqueous TEA for 1 h at 121° C. The Seaman hydrolysis was carried out as described below. Wall residues (2-4 mg) were added to 100 μl ultrapure water in a screw-capped borosilicate test tube, 300 μl conc. H₂SO₄ was added and the suspension was left for 3 h at room temperature with occasional vortexing. The suspension was then diluted with 6.6 ml ultrapure water and heated for 2 h at 100° C. After cooling, the solution was neutralised with saturated barium hydroxide. The resulting BaSO₄ precipitate was removed by centrifugation (1000×gₘₐₓ) for 5 min, the remaining supernatant was concentrated by rotor vaporisation and its monosaccharide composition determined.

Monosaccharide mixtures (5-15 μg) were applied to a Carbo-Pac PA10 column (Dionex, Sunnyvale, Calif., USA) and eluted isocratically with water at a flow rate of 1.5 ml/min. Sodium hydroxide (300 mM) was added to the column eluent continuously at a flow-rate of 0.5 ml/min, and the eluent was monitored using a pulsed amperometric detector (Dionex). Eluting monosaccharides were identified by comparison to retention times of sugar standards (fucose, rhamnose, arabinose, xylose, glucose, galactose and mannose) and quantified by integration of their respective peak areas and comparison to standard curves generated for each monosaccharide. The uronic acid content of each monosaccharide mixture was determined by the m-hydroxybiphenol assay (Blumenkrantz and Asboe-Hansen 1973).

Measuring Degree of Esterification

The degree of acetyl and methyl esterification was estimated by HPLC using the procedure desribed by Voragen et al. (1986).

Immunogold Labelling of Potato Tubers

Transformant and wild-type tuber cell walls were characterised essentially as described in Bush and McCann (1999). In brief: Low-temperature resin-embedded sections of glutaraldehyde-fixed tubers from wild type and transformant 13.1 were immunogold labelled with mAb LM5 (recognises 1,4-beta-galactan (Jones et al 1997)), silver-enhanced and analysed by reflection laser scanning confocal microscopy and electron microscopy, as described previously McCann et al 1992).

### EXAMPLE 1

A General Set of DNA Constructs for Plant Transformation

DNA Construct pPGB121s-new

The granule bound starch synthase promoter region from the vector pPGB121s (a kind gift from R. Visser) was amplified by the polymerase chain reaction (PCR) with primers 5′GATTACGCCAAGCTTTAACG3′ (SEQ ID NO:1) and 5′GGTTTGTCGACGAAATCAGAAATAATTGGAGG3′ (SEQ ID NO:2) introducing a HindIII site 5′ and a Sa/I site in the 3′ end of the PCR product. In addition, the PCR approach deleted a spurious translational start codon in the GBSS 5′ untranslated region. The product was then purified by agarose-gel electrophoresis and ligated into HindIII/Sa/I-cleaved pGUSNos (a gift from L. Sander) producing pGBSS-GUSNos. The GBSS promoter fragment was then excised from pGBSS-GUSNos with HindIII and XbaI, purified, and subsequently cloned in HindIII/XbaI digested pBl121 (Datla et al., 1992), generating the vector pPGB121-new. This vector was digested with SmaI and SacI in order to remove the GUS coding region. Subsequently, the SacI overhang were blunted with Klenow fragment and the vector closed by ligation resulting in pPGB121s-new.

DNA Construct pPGB121s-B

The vector pPGB121s-new was digested to completion with Sa/I and BamHI and purified by agarose gel-electrophoresis. A polylinker produced by annealing of two synthetic oligonucleotides with the sequences 5′ TCGACCGGTACCTAGGCCCGG ′3 (SEQ ID NO 3) and 5′ GATCCCGGGCCTAGGTACCGG 3′ (SEQ ID NO:4) was cloned in the Sa/I/BamHI cut vector. This step resulted in the vector pPGB121s-B harbouring an MCS with unique sites for AgeI, KpnI, AvrlI, SmaI and XmaI.

DNA Construct pGEd

The HindIII site was deleted in pPGB121s-new by digestion with HindIII followed by fill-in of the overhangs by reaction with Taq in the presence of nucleotides for 7 min. at 72° C. The resulting product was purified by agarose-gel electrophoresis and closed by ligation, generating pPGB121s-new-DHindIII. A novel polylinker produced by annealing of two synthetic oligonucleotides with the sequences 5′TCGACAAGCTTTCTAGAGCCTCGAGG3′ (SEQ ID NO:5) and 5′ GATCCCTCGAGGCTCTAGAAAGCTTG3′ (SEQ ID NO:6) was cloned into the remaining part of the original multiple cloning site (MCS), generating the plasmid pGED. The novel MCS introduced restriction sites for HindIII, XbaI and XhoI.

DNA Construct pADAP

pGED was digested with Sa/I and BamHI and purified by agarose gel-electrophoresis. A novel polylinker produced by annealing of two synthetic oligonucleotides with the sequences 5′TCG ACC GGT ACC AAG CTT GCG GGC TCT AGA CTC GAG CCT AGG CCC GG′ (SEQ ID NO:7) and 5′ GAT CCC GGG CCT AGG CTC GAG TCT AGA GCC CGC AAG CTT GGT ACC GG′ (SEQ ID NO:8) was cloned into the remaining part of the original MCS of pGED, generating the plasmid pADAP. The novel MCS introduced restriction sites for AgaI, KpnI, HindIII, XbaI, XhoI, AvrlI and SmaI.

DNA Construct pPGB121s-B-B33

The vector pBINB33 (a kind gift from L. Willmitzer) was digested with EcoRI and purified by agarose gel-electrophoresis. Following a linker encoding a HindIII site produced by annealing of the oligonucleotides 5′ AATTCAAGCTTG 3′ (SEQ ID NO:9) and 5′ AATTCAAGCTTG 3′ (SEQ ID NO:10) was cloned in the EcoRI cut pBINB33 vector. This step resulted in generation of the vector pBINB33-EHE. In order to isolate a fragment harbouring the patatin B33 promoter the pBINB33-EHE vector was digested with HindIII and Sa/I. The resulting fragment was cloned in HindIII/Sa/I digested pPGB121s-B, generating the plasmid pPGB121s-B-B33.

### EXAMPLE 2

Specific DNA Constructs for Plant Transformation Harbouring a Polysaccharide-modifying Enzyme Targeted for the Apoplast

DNA Construct pGED-GAL

The 1,3 kb cDNA encoding an Aspergillus aculeatus galactanase was excised from the vector pC1G1 (a generous gift from S. Kauppinen) by digestion with HindIII and XbaI, purified and cloned in the corresponding sites in the pGED MCS, creating the expression cassette: GBSS promoter, endo-galactanase, nopaline synthase terminator. This DNA construct is referred to as pGED-GAL.

Construction of pGED-ARA

A 1,2 kb cDNA encoding an Aspergillus aculeatus arabinanase was excised from the vector pC1A4 (a generous gift from S. Kauppinen) by digestion with HindIII and XbaI, purified and cloned in the corresponding sites in the pGED MCS giving rise to the plasmid pGED-ARA.

DNA Construct pADAP-ARA

The 1.2 kb fragment encoding the Aspergillus aculeatus arabinanase was isolated from pGED-ARA by digestion with HindIII/Sa/I and cloned into pre-cut pADAP. This step resulted in the vector pADAP-ARA.

DNA Construct pADAP-GAL

The 1.3 kb fragment encoding the Aspergillus aculeatus galactanase was isolated from pGED-GAL by digestion with HindIII/Sa/I and cloned into pre-cut pADAP. This step resulted in the DNA construct pADAP-GAL.

DNA Construct pPGB121s-B-B33-ARA

The 1.2 kb fragment encoding the Aspergillus aculeatus arabinanase was isolated from pADAP-ARA by digestion with KpnI and SmaI. The fragment was then cloned in pre-cut pPGB121s-B-B33. This step generated the DNA construct pPGB121s-B-B33-ARA.

DNA Construct pPGB121s-B-B33-GAL

The 1.3 kb fragment encoding the Aspergillus aculeatus galactanase was isolated from pADAP-GAL by digestion with KpnI and SmaI. The fragment was then cloned in pre-cut pPGB121s-B-B33, creating the expression cassette: patatin B33 promoter, endo-galactanase, nopaline synthase terminator. This step generated the DNA construct pPGB121s-B-B33-GAL.

DNA Construct pPGB121s-new-RHGB

The plant transformation vector pPGB121s-new was digested with the restriction enzyme Sa/I, blunt-ended with Klenow enzyme, and after heat-inactivation of the two enzymes further digested with the restriction enzyme XbaI. The vector pYES2/RHGB containing the cDNA clone encoding the Aspergillus aculeatus rhamnogalacturonan lyase was digested with the restriction enzyme BamHI, blunt-ended with Klenow enzyme, and after heat-inactivation of the two enzymes further digested with the restriction enzyme XbaI. The treated vector and cDNA insert were purified after agarose gel electrophoresis and ligated, creating the DNA construct pPGB121s-new-RHGB.

### EXAMPLE 3

Designing Constructs for Targeting the Gene Product to Intracellular Compartments.

DNA Construct pADAP/ARA-KDEL

The arabinanase encoding cDNA was amplified with the primers ACAGCTCAACAAGTGGTAAC (GBSSpro primer, SEQ ID NO:11) and GACTTCTCGAGTGGCTGG-CCTGTTGTGAAGGATGAACTTTAGTCTAGAAATGCTC (ARA-KDEL primer2, nucleotides encoding the KDEL ER retention signal is indicated with italics and an engineered stop codon is indicated in bold, SEQ ID NO:12) using the vector pGED/ARA as template. The resulting product was then cloned in the vector pCR2.1-TOPO as described by the manufacturer (Invitrogen). This step resulted a mixture of the vectors pCR2.1-TOPO/ARA-KDEL1 and 2 with the ARA-KDEL product in different orientations.

In order to minimise the possibility for later expression problems due to errors introduced during PCR, the majority of the ARA-KDEL coding region was swapped with the coding region originating from the original ARA cDNA included in the yeast expression vector pC1A4. The vectors pCR2.1-TOPO/ARA-KDEL1 and 2 was. cleaved with Sa/I and XbaI and the resulting KDEL encoding fragment cloned into SalI/XbaI-cut pC1A4. This resulted in generation of the vector pYES2.0/ARA-KDEL. The ARA-KDEL fragment was then excised from pYES2.0/ARA-KDEL with HindIII and XbaI and cloned in HindIII/XbaI digested pADAP. This generated the vector pADAP/ARA-KDEL.

DNA Construct pPGB121s-B-B33-ARA-KDEL

The ARA-KDEL fusion was excised from pADAP/ARA-KDEL with KpnI and SmaI and the fragment purified by agarose gel-electrophoresis. The resulting fragment was cloned into KpnI/SmaI digested pPGB121s-B-B33. This step generated the vector pPGB121s-B-B33/ARA-KDEL.

DNA Construct pPGB121s-B-B33-ARA-KDEL

The ARA-KDEL fusion was excised from pADAP/ARA-KDEL with KpnI and SmaI and the fragment purified by agarose gel-electrophoresis. The resulting fragment was cloned into KpnI/SmaI digested pPGB121s-B-B33. This step generated the vector pPGB121s-B-B33/ARA-KDEL.

DNA Construct pADAP/ST-ARA

The glycosyltransferase alpha-2,6-sialyltransferase (ST) is a Type II membrane protein localised to the Golgi apparatus. The first 44 amino acids of this protein have previously been shown to direct Golgi retention of a fused marker protein, lysozyme (Munro 1991). In addition, Golgi targeting of GFP has been accomplished by fusion of the 52 N-terminal amino acids of ST (Boevink et al. 1998).

To this end, a construct harbouring a fusion between the 52 N-terminal amino acids of ST and the mature form of the endo-arabinanase has been produced as follows. The ST region of relevance was amplified by PCR with primers (ST SOE FWD, ST specific sequences underlined) 5′ GACGAAGCTTATGATTCATACCAACTTG 3′ (SEQ ID NO:13) and (ST SOE REV, ST specific sequences underlined) 5′ GGAGCCGGGGTTGGCGTA-GGCCACTTTCTCCTGGCTC 3′ (SEQ ID NO:14). The plasmid pST-MYC used as a template was a kind gift from Søren Møgelsvang. The mature part of the arabinanase was then amplified with primers (ARA SOE FWD, arabinanase specific sequences in bold) 5′GAGCCAGGAGAAAGTGGCCTACGCCAACCCCGGCTCC 3′ (SEQ ID NO:15) and (ARA SOE REV, arabinanase specific sequences in bold) 5′ CAGTCTAGACTACACAACAGGCCAGCC3′ (SEQ ID NO:16). The two products were purified by agarose gel-electrophoresis and subsequently fused by sequence overlap extension PCR (Higuchi 1988) resulting in fusion of the 52 N-terminal amino acids of the ST to the mature part of the arabinanase (302 amino acids). The fusion product was purified by agarose-gel electrophoresis digested with HindIII and XbaI and cloned in HindIII/XbaI digested pADAP. The resulting plasmid was named pADAP/ST-ARA.

### EXAMPLE 4

Design of Constructs Harbouring Two Genes

DNA Constructs pGED/double/1 and pGED/double/2

The vector pPGB121s-B was cleaved with HindIII and EcoRI. The resulting fragment harbouring the GBSS-Tnos expression cassette was end-filled with Taq and the resulting product was cloned in the vector pCR2.1-TOPO as described by the manufacturer (Invitrogen). This step generated the vectors pCR2.1-TOPO/GBSS-nosterm1 and pCR2.1-TOPO/GBSS-nosterm 2. Each vector was digested with EcoRI and the resulting fragment purified by agarose-gel electrophoresis. The fragment was cloned in the vector pGED which had been digested with EcoRI and dephosphorylated with alkaline shrimp phosphatase as described by the manufacturer (Boehringer Mannheim). This step resulted in the vectors pGED/double/1 and pGED/double/2 harbouring two GBSS promoters followed by multiple cloning sites carrying Sa/I, HindIII, XbaI, XhoI, BamHI (MCS1) and Sa/I, AgeI, KpnI, AvrlI, SmaI, XmaI and BamHI sites (MCS2).

DNA Construct Harbouring Genes Encoding a Remodelling and an Auxiliary Enzyme, pPGB121s-new-RHGB-RHA1:

The vector pBI221 (Clontech) was digested with the restriction enzyme EcoRI, dephosphorylated, phenol/chloroform extracted and precipitated with alcohol. The phosphorylated oligonuleotide P-AATTAAGCTT (SEQ ID NO:17) was self-annealed and ligated into the EcoRI restricted pBI221 vector, creating the vector pBI221-2HindIII.

The vector pBI221-2HindIII was digested with the restriction enzyme SacI, blunt-ended with T4 DNA polymerase, and after heat-inactivation of the two enzymes further digested with the restriction enzyme BamHI. The treated vector ( pBI221-2HindIII*B*Sb) was purified after agarose gel electrophoresis.

The vector pGEM-7Z/RHA1 containing the cDNA clone encoding the Aspergillus aculeatus rhamnogalacturonan acetyl esterase was digested with the restriction enzyme XbaI, blunt-ended with Klenow enzyme, and after heat-inactivation of the two enzymes further digested with the restriction enzyme BamHI. The treated cDNA insert was purified after agarose gel electrophoresis and ligated into the gel-purified vector part pBI221-2HindIII*B*Sb, creating the vector pBI221-2HindIII-RHA1. The vector pBI221-2HindIII-RHA1 was digested with the restriction enzyme HindIII and the expression cassette containing the 35S promoter-rhamnogalacturonan acetyl esterase, nopaline synthase terminator was purified after gel electrophoresis.

The DNA construct pPGB121s-new-RHGB was digested with the restriction enzyme HindIII, de-phosphorylated, purified after gel electrophoresis, and ligated with the purified HindIII fragment containing the expression cassette: :35S promoter, rhamnogalacturonan acetyl esterase, nopaline synthase terminator, creating the DNA construct pPGB121s-new-RHGB-RHA1.

DNA Constructs pGED/apoGal/empty/1 and pGED/apoGal/empty/2

The construct pGED/GAL is digested with HindIII/XbaI and the resulting GAL encoding fragment is isolated by agarose-gel electrophoresis The vectors pGED/double/1 and pGED/double/2 are cleaved with HindIII and XbaI and purified by agarose-gel electrophoresis followed by cloning of the GAL encoding fragment. These steps generate two vectors designated pGED/apoGAL/empty/1 and pGED/apoGAL/empty/2, respectively. Hence, the two vectors have been equipped with an apoplastic targeted galactanase between the GBSS promoter and the Nos terminator in the first GBSS/Nos terminator cassette.

Design of ‘Self-processing’ Double DNA Constructs

The 1.3 kb cDNA fragment encoding the Aspergillus aculeatus endo-galactanase is isolated from one of the previously constructed vectors and cloned in between the GBSS promoter and the Nos terminator in one of the two GBSS/Nos terminator cassetes present in pGED/double/1 or 2, thus creating the apoplastic expression cassette GBSS promoter-endo-galactanase-Nos terminator. These vectors are termed pGED/apoGAL/1 and 2, and are the starting vectors used for the design of all double DNA constructs. The second GBSS promoter. Nos terminator cassette is used for the construction of expression cassettes designed for delivery of cell wall modifying enzymes, to the cytoplasm, the endoplasmic reticulum (ER) or the plant vacuole. The enzyme is preferentially an enzyme whose action post harvest releases the product of interest from the pulp cell walls. In the following Example 12 self-processing using as non-limiting examples the endo-galactanase as the remodelling enzyme, and endo-PG as the excising enzyme is demonstrated. Vector constructs introducing the endo-PG and targeting it for various subcellular locations are prepared as outlined:

Cytoplasmic targeting. For cytoplasmic targeting, endo-PGs from plants, bacteria or fungi with high stability in a neutral pH environment are preferred. The targeting to the cytoplasm is accomplished by removing the sequence from the endo-PG cDNA encoding the endo-PG signal peptide, thus abolishing its transport across the endoplasmic reticulum. If the N-terminus of the mature protein is not known from protein sequences, the cleavage site for the signal peptide is determined empirically using computer programs optimised for this purpose. The cDNA encoding the mature endo-PG protein is then cloned in between the GBSS promoter and the Nos terminator in the second GBSS/Nos terminator cassette in the constructs pGED/apoGal/empty/1 and pGED/apoGal/empty/2.

ER retention. For ER retention, endo-PGs from plants, bacteria or fungi with high stability in a slightly acidic to neutral pH environment are preferred. The targeting to the ER is accomplished by incorporating into the cDNA encoding the endo-PG a sequence encoding the tetrapeptide KDEL at the very end of the coding region, giving rise to an expressed protein with a KDEL extension at its C-terminal. The cDNA encoding the fusion protein is then cloned in between the GBSS promoter and the Nos terminator in the second GBSS/Nos terminator cassette in the vectors pGED/apoGal/empty/1 and pGED/apoGal/empty/2.

Vacuolar targeting. For vacuolar targeting, endo-PGs from plants, bacteria or fungi with high stability in an acidic environment are preferred. Targeting to the vacuole can e.g. be accomplished by attachment of a nucleotide sequence encoding a signal sequence ensuring translocation of the hybrid into the ER followed by a vacuolar signal sequence which e.g. comprises the amino acids NPIRL (an example of an N-terminal propeptide (NTPP)). Alternatively, the NPIRL extension or the like is fused to the C-terminal end of the protein in question while a signal sequence ensuring translocation into the ER is fused to the N-terminal part of the protein destined for vacuolar targeting (Koide et al. 1997).

An example of a protein which harbours an NTPP in its N-terminus is sweet potato sporamin. The N-terminal part of sporamin has been shown previously to direct proteins not normally found in the vacuole to this compartment when fused to the sporamin N-terminus. Hence, a nucleotide sequence encoding the N-terminal part of sporamin can be fused directly to the nucleotide sequence encoding an enzyme which hydrolyses the pectin backbone (i.e. with the signal peptide deleted) and thereby direct vacuolar targeting of the pectinase. The cDNA encoding the fusion protein is then cloned in between the GBSS promoter and the Nos terminator in the second GBSS/Nos terminator cassette. This vector is termed pGED/apoGALvacPG/1. Using single gene constructs, the following Examples 10 and 11 illustrate targeting of an endo-arabinanase to the ER and the vacuole, respectively.

### EXAMPLE 5

Remodelling Galactan Side Chains of Rhamnogalacturonan in Potato

Potato plants expressing a fungal (Aspergillus aculeatus) endo-galactanase (Christgau et al., 1995) under the control of the tuber-specific GBSS promoter have been generated, see Example 2. Apart from a low transformation efficiency the obtained plants displayed no altered phenotype compared to wild type plants. The lower frequency may indicate that the promoter is active during in vitro culture and that a high level of endo-galactanase activity at this very early stage of development may be lethal to the transformed cells. Endogenous endo-galactanase activity was almost undetectable in tuber extracts of wild type potato plants whereas for T_{11.1} and T_{13.1} the activity levels were 104 and 214 μmol galactose equivalents/min/g tuber fresh weight, respectively (Table 2). T_{11.1} and T_{13.1}were selected for analysis because of their cell wall phenotype as initially picked up by FTIR-spectrometry, see below.

Endo-galactanase activity in the transgenic plants was extracted quantitatively in low salt buffer, indicating that the enzyme is not tightly bound to the cell wall. Extracts from transgenic and wild type plants were also analysed by Western blot. All extracts having endo-galactanase activity contained a protein with a molecular mass of 38 kDa similar to that of isolated recombinant endo-galactanase.

During the isolation and extraction of cell walls, several precautions were taken to avoid enzymatic degradation of the pectic material by the introduced endo-galactanase as well as endogenous pectinases. Buffers containing sodium deoxycholate were used to denature and solubilise proteins, which were also diluted by several subsequent ice-cold extractions in buffers with a pH unfavourable for optimal activity of plant pectinases and in particular the introduced endo-galactanase. The endo-galactanase activity was also undetectable in the cell wall extraction buffer during preparation of cell walls. Fourier transform infrared microspectrometry was used for the initial selection of transformants. Exploratory principal component analysis (PCA) was able to clearly discriminate two of the transformants, T_{11.1} and T_{13.1}, from wild type. FIG. 1 shows the comparison between T_{13.1} and wild type, using the third and fifth Principal Component (PC) scores. These transformants were therefore selected for further analyses. The yield of cell wall material was lower in the transformants than in the wild type and so was the residual starch content (Table 2). Monosaccharide composition of the wall preparations revealed a dramatic reduction to ˜30% in galactose content in both transformants compared to wild type (Table 3). Standard variations were found to quite high in some monosaccharide determinations. However, the uncertainty is not associated with the transformed phenotype but almost invaribly with residual starch in the prepared cell walls. Galactosyl residues are present in different wall polysaccharides (hemicellulose, arabinogalactan proteins and RGI), but only RGI is known to contain beta-1,4-linked galactan, so our subsequent analysis of cell wall material focused on this polymer.

RGI is specifically extracted from walls by treatment with a combination of fungal EPG and PME. This EPG/PME treatment released nearly twice as much UA from the cell walls of transformed tubers compared to wild type (Table 3). The EPG/PME-solubilised pectin was analysed by size exclusion chromatography to separate the digest fragments according to molecular size. The RGI extracted by EPG/PME from wild type cell walls contained two major fractions as indicated by UA content and refractive index detection (FIG. 2): these were termed fraction A (molecular weight>500 kDa) and fraction C (molecular weight 0.2-8 kDa). EPG/PME extracts from T_{11.1} and T_{13.1} have a different profile from the wild type (FIG. 2), containing less of fraction A, substantially more of fraction C and in addition, fragments of ˜120 kDa (fraction B), not present in wild type extracts. The asterisk indicates a large peak due to the presence of sample buffer salts devoid of pectic material.

As determined by sugar analysis, (Table 3), fraction A of the wild type tuber contained high proportions of UA, rhamnose, arabinose and galactose with virtually no other monosaccharides, indicative of high molecular weight HGA and RGI polymers. However, while Fraction A from wild type tubers contained 64 mol % galactosyl residues, transgenic tubers contained only 15-20 mol %, suggesting a major reduction in the amount of galactan. The arabinosyl content of the transgenic tubers is slightly increased and the UA content is significantly higher. Interestingly, fucosyl residues could not be detected in the transgenic fraction A, but were present, albeit in a very low percentage, in wild type fraction A. (Fucosyl residues may be substituents of the galactan side-chains of potato RGI). The monosaccharide composition of fractions A and B from transgenic tubers was similar (Table 3). Fraction C from wild type and transformant tuber cell walls contained mainly HGA fragments, but some small molecular weight RGI fragments were also detected. The RGI fragments derived from the transgenic tubers, like those of fractions A and B, also had a lower galactosyl content (1.4-1.5 mol %) compared to those of wild type tubers (3.8 mol %). However, the relatively large amount of glucosyl residues may represent a contamination of the wall material with oligosaccharides released by the alpha-amylase treatment during cell wall preparation.

Enzymatic treatment of the walls with EPG/PME does not completely depectinate the walls. Therefore, additional sequential extractions were performed using sodium carbonate at 4° C. and room temperature (Table 3). De-esterification with carbonate solubilised very little pectin and, unlike the EPG/PME extraction, the yield from the wild type and transgenic cell wall preparations was similar. Size exclusion chromatography of the carbonate extracts resulted in two UA containing fractions (data not shown), one in the void volume (molecular weight>500 kDa) and one with a retention time equivalent to GalA, indicating the presence of monosaccharides or small oligosaccharides. The latter fraction could not be analysed for its monosaccharide composition because of the high salt concentration present in the sample. Monosaccharide analysis of the extracts containing the large polymers showed high proportions of arabinose, galactose, UA and rhamnose, indicative of RGI (Table 3) Significant amounts of glucosyl and xylosyl residues were probably derived from xyloglucan solubilised by the carbonate extractions. The carbonate extracts of the transgenic cell walls also showed reduced galactosyl content relative to wild type (18-19% versus 52%). Again, the arabinosyl content of the RGI present in carbonate extract is constant whereas the UA content is only higher in the cell wall preparations of T_{13.1} but not in T_{11.1}.

Pectins may remain in the wall after the enzymatic and carbonate buffer extractions. The monosaccharide compositions of cell walls before and after the sequential treatments with EPG/PME and carbonate were compared to quantify the efficiency at which pectins were solubilised (Table 3). No rhamnose could be detected in the walls after pectin extraction; although rhamnose is detected with only moderate sensitivity, this suggests that the RGI present in the unextracted walls was completely removed by the sequential extractions. However, the extracted walls of wild type and transformed tubers still contained galactose in similar quantities. This galactose most likely originates from other wall components than RGI, such as xyloglucan, which are known to contain beta-1,2-linked galactosyl residues, and cannot be hydrolysed by the endo-galactanase. Xyloglucans are only extracted by concentrated alkali or xyloglucanase treatment and would therefore be expected to be present in walls treated by the procedure used in this study. Interestingly, the UA content is more than 3-fold higher in the remaining walls of the wild type than in the transformants, indicating an increased extractability of pectin from the transformants.

To determine if 1,4-beta-D-galactan has been specifically hydrolysed by the endo-galactanase monoclonal antibody LM5 which recognises tetramers of 1,4-beta-D-galactan was used to immunogold-label wild type and T_{13.1} mature tuber tissue (FIG. 3).

FIG. 3 shows sections of wild type (A, C) and endo-galactanase-expressing (T_{13.1}) (B, D) potato tubers gold labeled with monoclonal antibody LM5, silver enhanced and viewed by reflection confocal scanning microscopy (A, B) and transmission electron microscopy (C, D). The walls of wild type parenchymal cells are strongly labeled (white in A, black particles in C), whereas in T_{13.1} tubers, the labeling density is greatly reduced and localized only to some cell corners (arrow heads in B) close to the plasma membrane (arrows in D). Asterisks represent spaces once occupied by starch granules. ML indicates the expanded middle lamella of these filled corners. Scale bars: A and B 100 mm, C and D 2mm.

To determine the architecural changes brought about in the rhamnogalacturonan (RG) molecule by the galactose removal, linkage pattern was determined according to Hakomori (1964) on isolated RG from T_{13.1} and the wild type control. The results summarised in table 1 below show the expected increase in terminal galactosyl residues acompanied by very significant reductions in severaltypes of internal galactosyl residues with the internal beta-1,4-galactosyl residue reduced to about one ninth of the wild type level and many of the lessabundant linkages reduced to below the detection limit in the transformant.

From these results it can be concluded that all RGI polymers isolated from transgenic walls had a significantly reduced galactose content, showing that the secreted enzyme was active in the walls and hydrolysed most galactan side chains of RGI. Immunolocalization indicated that the removal of substrate for the galactanase was nearly completes leaving hairy region galactans so short that neither antibody LM5 nor the galactanase could bind to them. Linkage analysis corroborate these results and demonstrate that not only has the composition been changed but a new rhamnogalacturonan architecture has been produced. Electron micrographs indicate the occurrence of small amounts of longer galactans on the side of the walls facing the plasma membrane (the most recently synthesised part of the wall), suggesting that deposition of newly synthesised galactans competes with removal of galactans, albeit with removal being the dominant process. This is the first demonstration of remodelling of monosaccharide profile and linkage pattern of a plant cell wall polysaccharide. The results are summarised in Table 1:

TABLE 1
Glycosidic linkage abundance (mol %) in cell walls of wild type
and transgenic potato

| Sugar | Wild type potato | Transgenic potato T_{13.1} |
|---|---|---|
| 2-Rha | 4.0 | 8.1 |
| 2,4-Rha | 7.7 | 13.6 |
| ^{ab}t-Araf | 4.1 | 4.1 |
| 5-Araf | 28.1 | 40.1 |
| 2,3,5-Araf | — | 2.9 |
| t-Gal | 7.2 | 13.9 |
| 3-Gal | 3.3 | 4.7 |
| 4-Gal | 36.1 | 4.0 |
| 3,4-Gal | 1.0 | — |
| 2,4-Gal | 1.4 | — |
| 4,6-Gal | 2.5 | — |
| t-xyl | 0.7 | 1.4 |
| 4-Xyl | 0.8 | 1.6 |
| ^{c}4-Glc | 2.8 | 5.6 |

^{a}t denotes terminal residues
^{b}Araf denotes furanose form of arabinosyl residues
^{c}Most of the 4-Glc is taken to be starch contamination)

### Conclusions

All RGI polymers isolated from transgenic walls had a significantly reduced galactose content, showing that the secreted enzyme was active in the walls and hydrolysed most galactan side chains of RGI. Immunolocalization indicated that the removal of substrate for the galactanase was nearly complete, leaving hairy region galactans so short that neither antibody LM5 nor the galactanase can bind to them. Electron micrographs indicate small amounts of longer galactans on the side of the walls facing the plasma membrane (the most recently synthesised part of the wall), indicating that deposition of newly synthesised galactans competes with removal of galactans, albeit with removal being the dominant process. This is the first demonstration of remodelling of monosaccharide profile or linkage pattern of a plant cell wall polysaccharide.

TABLE 2
Endo-galactanase activity and cell wall yield^{a}

| | Galactanase | | | | |
|---|---|---|---|---|---|
| | activity | | Residual | | |
| | (μmol galactose | Cell wall yield | starch | Extraction yield^{b} | |
| | eq./min/g fresh | (mg/g fresh | content of walls | (μg UA-equ./mg dry walls) | |
| | weight) | weight) | (mass %) | EPG/PME^{c} | Na₂CO₃^{c} |
| wild type | 1.6 | 13.1 ± 2.0 | 14.6 ± 5 | 71.9 ± 2.5 | 14.0 ± 1.4 |
| T_{11.1} | 104 | 8.6 ± 2.1 | 11.9 ± 6 | 115.5 ± | 11.5 ± 0.9 |
| | | | | 20.7 | |
| T_{13.1} | 214 | 9.5 ± 2.5 | 9.1 ± 5 | 142.2 ± | 20.3 ± 6.5 |
| | | | | 14.3 | |

^{a}Data (± SD) is the average of three independent experiments
^{b}Determined by the m-hydroxybiphenol assay
^{c}Extraction specifics are described in Materials and Methods

TABLE 3
Sugar compositions (mol %) of material obtained from transgenic and WT potato tubers^{a}

| | | | Fucose | Arabinose | Rhamnose | Galactose | Glucose | Xylose | Mannose | UA |
|---|---|---|---|---|---|---|---|---|---|---|
| Walls before | | wild | 0.3 ± 0.3 | 6.2 ± 2.7 | 1.4 ± 1.2 | 9.4 ± 1.5 | 53.0 ± 7.0 | 2.6 ± 0.2 | 0.4 ± 0.4 | 26.7 ± 1.7 |
| | | type | | | | | | | | |
| extraction | | T_{11.1} | 0.3 ± 0.1 | 6.8 ± 2.8 | 1.4 ± 1.2 | 3.3 ± 1.1 | 61.0 ± 5.6 | 3.0 ± 2.3 | 0.6 ± 0.5 | 23.5 ± 8.0 |
| | | T_{13.1} | 0.3 ± 0.1 | 4.8 ± 1.2 | 0.8 ± 0.4 | 2.6 ± 0.4 | 63.5 ± 5.9 | 1.3 ± 0.3 | 0.8 ± 0.4 | 25.9 ± 6.6 |
| EPG/PME | A | wild | 0.2 ± 0.2 | 15.8 ± 1.2 | 5.7 ± 2.0 | 64.6 ± 4.1 | 0.3 ± 0.2 | 0.4 ± 0.3 | nd^{b} | 13.0 ± 2.5 |
| | | type | | | | | | | | |
| extract | | T_{11.1} | nd | 30.3 ± 9.9 | 10.4 ± 8.4 | 20.5 ± 2.5 | 0.4 ± 0.4 | 2.4 ± 0.8 | nd | 36.0 ± 2.2 |
| | | T_{13.1} | nd | 25.1 ± 6.9 | 7.0 ± 4.5 | 15.8 ± 3.6 | 0.2 ± 0.3 | 1.4 ± 1.5 | nd | 50.5 ± 12.3 |
| | B^{c} | T_{11.1} | nd | 32.3 ± 1.1 | 7.8 ± 3.1 | 20.3 ± 0.5 | 0.3 ± 0.4 | 0.7 ± 0.6 | nd | 38.6 ± 4.3 |
| | | T_{13.1} | nd | 20.8 ± 7.8 | 5.5 ± 2.2 | 13.3 ± 3.9 | 0.4 ± 0.6 | 0.4 ± 0.8 | nd | 59.6 ± 11.8 |
| | C | wild | nd | 2.2 ± 0.7 | 2.5 ± 0.5 | 3.8 ± 1.5 | 24.8 ± 24.9 | 0.1 ± 0.2 | nd | 66.4 ± 22.8 |
| | | type | | | | | | | | |
| | | T_{11.1} | nd | 2.0 ± 0.6 | 2.5 ± 2.2 | 1.4 ± 0.6 | 28.9 ± 25.4 | 0.3 ± 0.3 | nd | 64.9 ± 24.3 |
| | | T_{13.1} | nd | 4.2 ± 2.8 | 1.1 ± 0.3 | 1.5 ± 1.0 | 9.5 ± 3.3 | 1.5 ± 2.6 | 0.4 ± 0.7 | 81.9 ± 10.3 |
| Na₂CO₃ | | wild | nd | 23.0 ± 7.9 | 7.1 ± 3.3 | 52.0 ± 10.9 | 5.2 ± 5.9 | 2.8 ± 2.8 | nd | 9.9 ± 3.7 |
| | | type | | | | | | | | |
| extract | | T_{11.1} | nd | 38.9 ± 13.7 | 13.6 ± 6.9 | 18.9 ± 3.3 | 5.5 ± 5.0 | 3.7 ± 1.3 | 1.5 ± 2.8 | 17.9 ± 8.3 |
| | | T_{13.1} | nd | 34.2 ± 14.9 | 9.7 ± 6.6 | 18.3 ± 4.6 | 3.4 ± 4.8 | 2.3 ± 2.1 | nd | 32.1 ± 28.3 |
| Walls after | | wild | nd | 4.2 ± 0.5 | nd | 2.4 ± 1.1 | 46.0 ± 5.9 | 15.6 ± 5.4 | 1.5 ± 1.9 | 30.2 ± 0.9 |
| | | type | | | | | | | | |
| extraction | | T_{11.1} | nd | 3.6 ± 0.2 | nd | 2.4 ± 1.5 | 73.3 ± 7.6 | 7.8 ± 5.9 | 5.8 ± 9.2 | 7.0 ± 2.9 |
| | | T_{13.1} | nd | 3.4 ± 1.1 | nd | 1.8 ± 0.5 | 74.2 ± 7.6 | 10.5 ± 6.4 | 0.3 ± 0.1 | 9.7 ± 1.0 |

^{a}Data (± SD) is the average of three independent experiments
^{b}not detectable
^{c}Fraction B was not present in EPG/PME extracts from WT plants (see FIG. 2)

### EXAMPLE 6

Galactanase Driven by the Patatin Promoter

Transgenic potato plants were produced essentially as described in Example 5 using the pPGB121 s-B-B33-GAL so that the galactanase would be driven by the patatin promoter rather than the GBSS-promoter. Analysis of gene expression has shown that expression was effectively confined to the tubers, and required higher concentrations of sucrose for induction in other organs as compared with the GBSS-promoter. Expression in tubers was not significantly different from that seen with the GBSS-promoter. Tubers can be subjected to immunological characterisation using the LM5 antibody to confirm that the cell wall phenotype in the tubers does not depend on the promoter driving expression of the galactanase gene.

### EXAMPLE 7

Potato Transformed to Express the Rhamnogalacturonan-lyase Targeted to the Apoplast

250 explants were used for transformation with Agrobacterium tumefaciens (see pPGB121s-new-RHGB and experimental procedures in Materials and Methods). This resulted in the generation of 21 individual transgenic lines, of which 18 were used for the present analysis. In general, the transgenic plants were normal and only some (18%) had a change in phenotype. Compared with the controls, these plants were smaller and also had smaller leaves. The leaves were thicker, rough and curled. Furthermore, these plants were darker compared to all other transgenics and the controls. All plants with an altered phenotype died before they produced tubers. In other experiments (transformations with other genes) we found similar phenotypes, suggesting that this is an effect of the transformation procedure itself (like polyploidisation) and not of a particular gene or construct. The normal looking plants appeared to produce tubers with an altered phenotype.

After initial analysis of DNA, RNA and protein level, interesting lines were propagated to generate more material for further analysis. All transgenic lines that had been successfully transformed (based on Kanamycin resistance) were subjected to Southern blot analysis. In fifteen out of eighteen plants, the rhg-lyase gene was detected. In all the other plants the gene is present in either one or two copies. Only those plants that actually produced tubers were analysed for expression of the transgene. Therefore only RNA, isolated from 11 different lines, was used for the Northern blot analysis. RNA gel blot hybridisation with a ribosomal potato DNA fragment as a probe showed equal amounts of RNA in each lane. Hybridisation of tuber RNA using the rhamnogalacturonan-lyase probe showed a clear transcript of the gene for most of the plants (nine out of eleven). Four of these nine can be designated as high expressors, while the other five show a lower expression level, among which a further differentiation could be made. The two that did not show any hybridisation also gave a negative result in the Southern blot analysis. We did not find a clear correlation between the number of copies (one or two) of the transgene and the RNA expression level. All the lines in which expression of the transgene could be detected were used for further analysis. Enzymatic characterisation of transgenic potato plants expressing the RG-lyase showed that 3 out of 9 RG-lyase transformants showed high lyase activity (complete degradation of the substrate into oligomers), whereas the others had moderate (1 out of 9), low (2) or very low (3) activity. Furthermore, a positive correlation was found between mRNA content and enzyme activity.

The sugar composition of the tubers of the RGL transformants were found to be rather similar to that of the control plants with regard to the substrate for the rhamnoglacturonan lyase. Table 4 below provides data on analysis of total cell walls from a high and a low expressing transformant and wild type potato tubers. A reduction from 1.5 to 1% rhamnose is indicative of a reduction in hairy region content.

TABLE 4
Composition of isolated cell wall, w/w % (upper panel) and mol %
(lower panel) of monosaccharides in total cell walls from cv Kamico
wild type and two RG-lyase expressing transformants
Sugar composition, starch and protein content in the
isolated cell wall material

| | wild type | High expressor | Low expressor |
|---|---|---|---|
| % w/w | | | |
| Protein | 8.8 | 10.3 | 8.6 |
| Starch | 3.7 | 1.2 | 1.4 |
| Total sugar | 75.0 | 71.0 | 71.0 |
| Mol % | | | |
| Rhamnose | 1.5 | 1.0 | 1.0 |
| Arabinose | 8.3 | 4.7 | 5.3 |
| Xylose | 2.5 | 4.1 | 4i1 |
| Mannose | 1.6 | 2.1 | 2.6 |
| Galactose | 22.0 | 4.4 | 5.8 |
| Glucose | 38.3 | 41.7 | 42.8 |
| Uronic Acid | 25.9 | 42.1 | 38.4 |
| DA | 35.0 | 18.0 | 19.0 |
| DM | 25.0 | 41.0 | 36.0 |

The above results might be surprising at first, especially when considering the altered phenotype of the tubers. The results can be explained as follows. The hairy regions are considered to interconnect homogalacturonan sequences in native pectin (Shols & Voragen 1996). The rhamnogalacturonan lyase attacks these interconnecting sequences, but cannot degrade the rhamnogalacturonan completely. Thus, a relatively small part of the hairy regions can escape from the cell wall as oligosaccharides released by endo rhamnogalacturonan lyase. Rhamnose (indicative of rhamnogalacturonan) is a relatively minor sugar in the cell wall. Galacturonic acid is much more abundant in the cell wall, but resides for the largest part in homogalacturonan. The new cell wall phenotype is indeed an architectural rather than compositional phenotype as can be described using immunolocalization of particular epitopes in the wall as was done in Example 5. Rhamnogalacturonan side chains consisting of galactans and arabinans rely on rhamnose residues for their attachments. It is noteworthy that there are very significant reductions in both sugars indicating major architectural changes in the hairy regions leading to a sparsely substitution with neutral sugar side chains. Immunolocalisation using arabinan and galactan antibodies in confocal microscopy corroborate the compositional analysis. Further these examinations reveal that paranchymal starch containing cells show very minor labelling for both epitopes whereas some cortical cells in the transformants label for galactans and arabinans in cell corner reinforcements.

### EXAMPLE 8

Targeting of Enzyme Activity to the Golgi Apparatus

Expressing and targeting a fungal arabinanase to the apoplast following the strategies described in Example 6 for the galactanase yields viable plants of both tobacco and potato. While the tobacco plants did not display any visible phenotype, most of the potato transformants had partly dysfunctional meristems. Using the GBSS-promoter to control the arabinanase, almost all leaf axil buds had non-functional meristems. A new unbranched plant architecture was thus created exemplifying the utility of the present invention also for controlling aspects of plant development.

Golgi-targeting of enzyme activity should be seen as an alternative to secretion to the apoplast (not as an intracellular site for storing enzymes in ‘self processing plant material’). The strength of this technology is to be able to interfere directly with polysaccharide biosynthetic processes occurring in the Golgi. The technology provides greater latitude in engineering options without compromising plant viability.

A glycosyl transferase alpha-2,6-sialyltransferase (ST) originating from rat has previously been shown to be targeted to the plant cell Golgi apparatus, both alone (Wee et al., 1998) and in truncated forms fused to lysozyme (Munro, 1991) or Green Fluorescent Protein (GFP) (Boevink et al., 1998). Hence, this protein has been evaluated for its ability to facilitate proper Golgi retention of various heterologous proteins. The ST is a type II membrane protein (i.e. with the amino terminus located in the cytosol) consisting of a signal sequence which is post-translationally cleaved of during transit into the ER, a short amino-terminal cytoplasmic domain, an uncleaved hydrophobic signal anchor directing Golgi localisation and a large catalytic lumenal domain (Weinstein et al., 1987). Accordingly, the internal signal anchor should be sufficient to retain any heterologous protein N-terminally fused to this part of the ST. This has been indicated by the work of Boevink et al. (1998) and Munro (1991), who fused 52 and 44 amino acids, respectively, of the N-terminal part of ST to chicken lysozyme (Munro 1991) and GFP (Boevink et al., 1998).

The rat sialyl tranferase therefore fulfills the general demands that are expected of a protein which is tightly bound to the Golgi membrane as opposed to proteins found in a soluble form in the Golgi apparatus. The soluble ones are expected to move through the Golgi and periodically reside at the plasma membrane, thereby coming in direct contact with the cell wall.

To this end, the rat sialyl transferase was fused to the mature part of the Aspergillus aculeatus arabinanase using recombinant PCR (Higuchi 1988) and the resulting fusion introduced into the expression vectors pADAP and pPGB121s-B-B33, thereby generating two constructs with the ST-ARA fusion under control of the GBSS and patatin promoter, respectively. The constructs have been transferred into Agrobacterium by electroporation and the bacterial hosts used for transformation of Nicotiana tabacum (L.) cv. Xanthi and Solanum tuberosum (L.) cv. Posmo.

Using differential centrifugation it was established that major portions of the enzyme reside in the microsomal fraction. Localisation to the Golgi was demonstrated using classical organelle separation methods (Ray et al., 1969, Gibeau and Carpita 1990).

Plants harbouring the ST-ARA fusion under the control of the GBSS-promoter was analysed. Expression of active enzyme was demonstrated in tuber extracts using the arabinanase plate assay. Western blotting was used to demonstrate. the association of the activity with a microsomal fraction, and following organelle separation, it could be demonstrated that the majority of the arabinanase was associated with Golgi vesicles, while no enzyme protein could be detected in the soluble apoplastic and cytoplasmic fractions, nor in the plasma membrane. Detectable, but lower levels were observed in the endoplasmatic reticulum which may well represent newly produced arabinanase in transit to the Golgi. Cross contamination of the endoplasmatic reticulum fraction with small amounts of Golgi vesicles cannot be excluded. Of primary importance is that the experiments demonstrate that the arabinanase has been transformed from a soluble to a membrane bound enzyme, and that the primary target is the Golgi apparatus.

Cell walls from these tubers were analysed for the monosaccharide composition of EPG/PME extracted hairy regions as was done in Example 5. The results are summarised in the below Table 5:

TABLE 5
Mol % sugars in hairy regions extracted from wild type and transformants
expressing an endo-arabinanase targeted to the Golgi

| Sugar residue | Wild type | Transformant 5.2 |
|---|---|---|
| arabinose | 7.9 | 3.8 |
| galactose | 62.9 | 68.8 |
| rhamnose | 10.7 | 13.6 |
| galacturonic acid | 13.1 | 12.7 |
| xylose | 0.9 | 1.1 |
| glucose | 4.5 | 7.4 |

To summarise, expression of the arabinanase targeted to the Golgi resulted in about 50% reduction in hairy region-bound arabinose. Labeling patterns with a an arabinan specific antibody observed by confocal microscopy corroborated the compositional data. This is the first example of targeted interference with complex polysaccharide biosynthetic processes in Golgi by any method.

### EXAMPLE 9

Double Construct with Auxiliary Enzyme

From in vitro studies it is known that the effect of rhamnogalacturonan-lyase is larger if it is used in combination with rhamnogalacturonan-acetyl esterase. Therefore, double constructs have been made to obtain expression of both these enzymes in the potato tubers, see pPGB121s-new-RHGB-RHA1 in Example 4. Potato plants have been transformed and 21 individual transgenic lines have been obtained. No important phenotypical changes were observed. However, we did expect a higher impact from the combination of these two enzymes as compared to the transformants in which only the rhg-lyase is present. As the lyase was found to be active on its own, a qualitatively similar phenotype is anticipated.

### EXAMPLE 10

Retaining a Polysaccharide-modifying Enzyme in the ER

Retention in the ER will be exemplified in the following because of its relevance for ‘self processing plant material’ as defined herein. In a preferred embodiment of the invention, an enzyme, typically a hydrolase or a lyase, which cleaves polysaccharide backbones is retained in the ER. Upon homogenisation of the plant material the enzyme is brought into contact with the cell wall and renders soluble a component to be recovered in the supernatant fraction. ER-retention is exemplified here with reference to arabinanase; other pertinent enzymes are listed in Example 12 below.

The arabinanase, SEQ ID NO:1 from WO 94/20611, was found to be toxic to potato, at least when secreted to the apoplast (Libiakova et al., 1999). Tobacco was used in the present case to demonstrate accumulation of a polysaccharide-modifying enzyme in active form in the ER. As detailed in Materials and Methods, the gene was a KDEL tagged variant of the endo-arabinanase in tobacco cv. Xanthi transformed with a pADAP-based construct harbouring an arabinanase-KDEL cDNA.

Extracts from leaves of transformed plants were evaluated for their enzyme accumulation using the plate assay (see Materials and Methods) and all successfully transformed plants also produced active arabinanase. The assay is semi-quantitative in nature but activity in crude leaf extracts was detectable (blue halos more than 1 mm wide around the punched wells holding the samples) within just 15 minutes (with some variation between transformants) indicating very significant enzyme activity. By differential centrifugation it can be demonstrated that an appreciable proportion of the enzyme is trapped in the microsomal fraction, indicating retention in the ER. Additionally the apoplastic fluid of tobacco leaves can be isolated using the method of Husted et al. (1995). Possible absence of appreciable arabinanase activity in the apoplastic fluid can be taken as evidence against missorting of the enzyme to the apoplastic space.

### EXAMPLE 11

Targeting an Enzyme to the Vacuole

As for retention in the ER, exemplification of vacuolar targeting shall be seen in the context of “self processing plant material”. A single gene-construct demonstrating targeting to an intracellular compartment has been presented in Example 3. Enzymes which are believed to be stable in the acidic environment of the vacuole may be stored in this organelle. Two general approaches are at hand: Equipping the gene of interest with a 5′-sequence from a gene whose product is accumulated there as is; or alternatively engineering the gene of interest with an N-terminal extension which will be cleaved off (partially or in toto) upon entry into the vacuole. The N-terminal portions of the vacuolar potato patatin protein (146 amino acids) (Sonnewald et al., 1991) and the sweet potato sporamin protein (111 amino acids) (Turk et al., 1997) are suitable candidates for this purpose.

In sweet potato sporamin, the vacuolar targeting information resides in the N-terminal part of the primary structure. Following cleavage of the N-terminal signal sequence, which ensures transport of the protein to ER, the pro-form is efficiently targeted to the vacuole where the pro-peptide is cleaved off (Koide et al., 1997). Unlike potato patatin, where the exact localization of the vacuolar targeting determinant is known to reside in the N-terminal but where the exact localisation is unknown, the vacuolar targeting signal in sporamin is well characterized. Accordingly, sporamin is used for targeting of heterologous proteins to the plant vacuole. Following translocation into the vacuole, the majority of the sporamin part of the fusion protein will be cleaved off, thereby resulting in a heterologous protein with a higher degree of authenticity. A similar approach with patatin will result in a permanent N-terminal extension, which may lead to misfolding and activity loss of the heterologous protein.

To this end, the vacuole-directing potato patatin CDR and 111 amino acids of the sweet potato sporamin coding region were cloned and sequenced. Constructs can be generated that harbour fusions between the N-terminal of sporamin/patatin and suitable endo-polygalacturonase; see the following example. Western blot analysis can be used to verify processing upon vacuolar entry (where applicable). Evaluation of transformants parallel to what has been described for ER-retained endo-arabinanase will show accumulation of active endo-PG in the vacuole.

### EXAMPLE 12

Self-processing Tubers Comprising Double Constructions Expressing Galactanase+Endo-polygalacturonase

A double construct harbouring the galactanase destined for secretion into the apoplast as well as an endo-PG of fungal or plant origin targeted for internal storage can be prepared, see “Design of “Self processing” DNA constructs” in Example 4. The galactanase should be regarded as a place holder for any enzyme catalysing a desirable pectin tailoring in vivo. Plant and fungal endo-polygalacturonases are used to exemplify ‘self processing plant material’ in this example. However, it should be borne in mind that also lyases of plant or microbial origin are similarly useful in this context.

Fungal endo-polygalacturonases are selected for their stability properties (e.g. with the acidic vacuolar environment in mind), with regard to their sensitivity to homogalacturonan decoration (acetylation in particular) and with regard to pH-optimum for catalysis. Polygalacturonases I, II and III from Aspergillus aculeatus, accession number AFO74213, WO 94/14952 and WO 94/14952, cover much of the range in biochemical properties of interest, but other polygalacturonases can also be used.

Comparison of higher plant endo-PG protein sequences has revealed two distinct types of endo-PGs. Those that posses a propeptide located in between the signal peptide and the mature protein and those that do not posses such a propeptide. The function of the propeptide has yet to be established.

Targeting of the double construct harbouring non-secreted endo-PG of the type which does not posses a propeptide (modified endo-PG A,B, and C) can be done as already exemplified. for the targeting of the endo-arabinanase to the ER (Example 10) and the vacuole (B and C) (Example 11). In this case, however, accumulation of the gene products in the cytoplasm is also considered (A). Wegener et al. (1996) describe how an Erwinia carotovora pectate lyase was accumulated in the cytoplasm in both leaves and tubers of transgenic potatoes without detectable phenotypic changes. Expressed thermostable bacterial cellulases have also been successfully accumulated in the cytoplasm of transgenic plants. Ziegelhoffer and co-workers produced the E2 and E3 cellulases from Thermomonospora fusca in transgenic alfalfa, potato and tobacco, although in very limited amounts (Ziegelhoffer et al., 1999). No phenotypic effect of cellulase expression was detected.

Whereas KDEL, as described previously, retains an already translocated protein in the ER, the signal peptide is responsible for the actual translocation. Thus, in order to obtain cytoplasmic accumulation of the enzymes in question, the signal peptide has to be removed prior to expression in the plant host. In case the signal peptide cleavage site has not been determined by protein sequencing, determination of the most likely cleavage site can be made, for example using the neural network SignalP (Nielsen et al, 1997).

The physical removal of the signal peptide is done at the DNA level by the use of PCR with primers specific for the mature part of the enzyme in question. This approach also ensures the introduction of a start codon and sequences for optimal translation of the mature part of the enzyme destined for cytoplasmatic accumulation.

Representative plant endo-PGs without propeptide include, but are not limited to, AF128266: PG1 from Glycine max and U70480:TAPG2 from tomato.

Targeting of non-secreted endo-PG of the type which posses a propeptide (modified endo-PG D-I) to the cytoplasm, the ER, or the vacuole should for each compartment be accomplished by two versions of modified endo-PG. One series (D, F and H) where the three modifications described in the previous section leave the propeptide sequence intact in front of the mature protein and one series (E, G and I) of three where the propeptide is removed, resulting in a total of six different constructs (D-I) covering three different localisations with or without propeptide included. Examples of plant endo-PGs with propeptide are X9500: RDPG1 from oilseed rape and P35336 from kiwi fruit. Analysis according to Example 6 can be used to verify that the wall phenotype resulting from galactanase expression is as expected from the results of Examples 5 and 6. In addition, expression and accumulation of functional endo-polygalacturonase will be established by Northern and Western blot analysis as well as assays of polygalacturonase activity.

### REFERENCES

Baron-Epel, O., Gharyal, P. K. & Schindler, M. (1988) Pectins as mediators of wall porosity in soybean cells Planta 175:389-395.

Blumenkrantz, N. & Asboe-Hansen, G. (1973) Anal. Biochem. 54:484489.

Boevink, P., Oparka, K., Santa Cruz, S., Martin, B., Betteridge, A., and Hawes, C. (1998). Stacks on tracks: the plant golgi apparatus traffics on an actin/ER network. Plant J. 15:441447

Boevink, P., Oparka, K., Santa Cruz, S., Betteridge, M. B., Hawes, C. (1998) Stacks on tracks: The plant golgi apparatus traffics on an ER/actin network. Plant J. 15:441447

Braccini, I., Grasso, R. P., Pérez, S., (1999) Conformational and configurational features of acidic polysaccharides and their interactions with calcium ions: a molecular modeling investigation. Carbohydr. Res. 317:119-130.

Bush, M. S., McCann, M. C. (1999) Pectic epitopes are differentially distributed in the cell walls of potato (Solanum tunberosum) tubers. Physiol. Plant 107:201-213.

Carpita, N. C. & Gibeaut, D. M. (1993) Structural models of primary cell walls in flowering plants: consistency of molecular structure with the physical properties of the walls during growth. Plant J. 3:1-30.

Chanliaud, E., Gidley, M. J. 1999. In vitro synthesis and properties of pectin/Acetobacter xylinus cellulose composites. Plant J. 20:25-35.

Chen, L., Carpita, N. C., Reiter, W. -D., Wilson, R. H., Jeffries, C. &McCann, M. C. 1998. A rapid method to screen for cell-wall mutants using discriminant analysis of Fourier transform infrared spectra. Plant J. 16:385-392.

Christgau, S., Sandal, T., Kofod, L. V. & Dalbøge, H. (1995) Expression cloning, purification and characterization of a b-1,4-galactanase from Aspergillus aculeatus. Curr. Genet 27:135-141

Côtè, F. & Hahn, M. G. (1994) Oligosaccharins: structures and signal transduction Plant Mol. Biol. 26:1379-1411.

Daas, P. J. H., Arisz, P. W., Schols, H. A., de Ruiter, G. A., Voragen, A. G. J. (1998) Analysis of partially methyl-esterified galacturonic acid oligomers by high-performance anion-exchange chromatography and matrix-assisted laser desorption/ionization time-of-flight mass spectrometry. Anal. Biochem. 257:195-202.

Datla, R. S., Hammerlindl, J. K., Panchuk, B., Pelcher, L. E. & Keller, W. (1992) Gene 122, 383-384. [published erratum appears in Gene 129:311.

Dhugga, K. S., Tiwari, S. C., Ray, P. M. (1997) A reversibly glycosylated polypeptide (RGP1) possibly involved plant cell wall synthesis: Purification, gene cloning, and trans-Golgi localization Proc Natl Acad Sci USA 94:7679-7684

Edwards, G. A., Hepher, A., Clerk, S. P. & Boulter, D. (1991) Pea lectin is correctly processed, stable and active in leaves of transgenic potato plants. Plant Mol. Biol. 17:89-100

Essl, D., Dimberger, D., Gomord, V., Strasser, R., Faye, L., Glossl. J., Steinkellner, H. (1999) The N-terminal 77 amino acids from tobacco N-acetylglucosaminyltransferase I are sufficient to retain a reporter protein in the Golgi apparatus of Nicotiana benthamiana cells. FEBS Lett 453(1-2):169-73

Fry, S. C. (1986) Cross-linking of matrix polymers in the growung cell walls of angiosperms. Ann. Rev. Plant Physiol. 37:165-86.

Gibeaut, D. M., Carpita, N. C. (1990) Separation of membranes by flotation centrifugation for in vitro synthesis of plant cell wall polysaccharides Protoplasma 156:82-93

Hakomori S I (1964) A rapid permethylation of glycolipid and polysaccharide catalyzed by methylsulfinyl carbanion in dimethyl sulfoxide. J Biochem 55:205-208

Herbers, K., Wilke, I., Sonnewald, U. (1995) A thermostable Xylanase from Clostridium thermocellum expressed at high levels in the apoplast of transgenic tobacco. Bio/technology 13:63-66

Higuchi, R., Krummel, B., and Saiki, R. K. (1988). A general method of in vitro preparation and specific mutagenesis of DNA fragments: Study of protein and DNA interactions. Nucleic acids research 16:7351-7367.

Horsch, R. B., Fry, J. E., Hoffmann, N. L., Eichholtz, D., Rogers, S. G., and Fraley, R. T. (1985). A simple and general method for transferring genes into plants. Science 227:1229-1231.

Huisman, M. M. H.; Schols, H. A.; Voragen, A. G. J. (1999) Enzymatic degradation of cell wall polysaccharides from soybean meal.Carbohydrate Polymers 38:299-307

Husted, S., Schjoerring, J. K. (1995). Apoplastic pH and ammonium concentration in leaves of Brassica napus L. Plant Physiol. 109:1453-1460.

Kempin, S. A., Liljegren, S. J., Block, L. M., Rounsley, S. D. Yanofsky, M. F. (1997) Targeted disruption in Arabidopdis. Nature 389:802-803.

Jarvis, M. C., Hall, M. A., Threlfall, D. R. & Friend, J. (1981) The polysaccharide structure of potato cell walls: chemical fractionation. Planta 152:93-100.

Jones, L., Seymour, G. B. & Knox, J. P. (1997) Localization of pectic galactan in tomato cell walls using a monoclonal antibody specific to (1,4)-b-D-galacatan. Plant Physiol 113:1405-1412.

Koide, Y., Hirano, H., Matsuoka, K., and Nakamura, K. (1997). The N-terminal propeptide of the precursor of sporamin acts as a vacuole-targeting signal even at the C terminus of the mature part in tobacco cells. Plant physiol 114:863-870.

Kuipers, A. G. J., Jacobsen, E., and Visser, R. G. F. (1994) Formation and deposition of amylose in the potato tuber starch granule are affected by the reduction of granule-bound starch synthase gene expression. /tge Okabt/cekk 6:3-52

Landsmann, J., Uhrig, H. (1985) Somoclonal variation in Solanum tuberosum detected at the molecular level. Theor. Apll. Genet. 71:500-505

Lau, J. M., McNeil, M., Darvill, A. G. & Albersheim, P. (1985) Structure of the backbone of rhamnogalacturonan I, a pectic polysaccharide in the primary cell walls of plants. Carbohydrate Res. 137:111-125..

Lever, M. 1972. A new reaction for calorimetric determination of carbohydrates.—Analytical Biochemistry. 47:273-9

Libiakova, G., Skjøt, M., Sørensen, S., Jørgensen, B., Ulvskov, P. (1999). Influence of constructs harbouring an arabinanase encoding cDNA on potato transformation efficiency. “From Cell to Crops”. The Third International Symposium in the Series: Recent Advances in Plant Biotechnology. September 1999, Stara Lesna, Slovak Republic.

Mayer, F. (1998) Potato pulp: propeties, physical modification and applications. Polymer Degrad. and Stability 59:231-235.

McCann, M. C. &Roberts, K. (1994) Changes in cell wall architecture during cell elongation. Journal of Experimental Botany 45:1683-1691.

McCann, M. C., Hammouri, M. K., Wilson, R. H., Belton, P. S. & Roberts, K. (1992) Fourier transform infrared microscopy is a new way to look at plant cell walls. Plant Physiol. 100:1940-1947.

Moloshok, T., Pearce, G., and Ryan, C. A. (1992). Oligouronide signaling of proteinase inhibitor genes in plants: structure-activity relationships of Di- and trigalacturonic acids and their derivatives. Arch. Biochem. Biophys. 294:731-734.

Munro, S. (1991). Sequences within and adjacent to the transmembrane segment of alpha-2,6-sialyltransferase specify Golgi retention. EMBO J 10:3577-88.

Nielsen, H., Engelbrecht, J., Brunak, S., and von Heijne, G. (1997). Identification of procaryotic and eukaryotic signal peptides and prediction of their cleavage sites. Prot. Engineering 10:1-6.

O'Neill, M. A., Albersheim, P. & Darvill, A. G. (1990) in The pectic polysaccharides of primary cell walls, ed. Dey, P. M. pp. 415-441.

O'Neill, M. A., Warrenfeltz, D., Kates, K., Pellerin, P., Doco, T., Darvill, A. G. & Albersheim, P. (1996) Rhamnogalacturonan-II, a pectic polysaccharide in the walls of growing plant cell, forms a dimer that is covalently cross-linked by a borate ester . In vitro conditions for the formation and hydrolysis of the dimer J. Biol. Chem. 271:22923-22930.

Paulsen, B. S. (ed.) (2000) Bioactive carbohydrate Polymers. Proceedings of the phytochemical society of Europe. Kluwer academic publishers, Dordrecht, Boston, London.

Ray, P. M., Shiniger, T. L., Ray, M. M. (1969) Isolation of [symbol] -glucan synthetase particles from plant cells and identification with golgi membranes Proc Natl Acad Sci USA 64:605-612

Rogers, S. O., Bendich, A. J. (1988) Extraction of DNA from plant tissues. In: Plant Molecular Biology Manual. S. B. Gelvin & R. A. Schilperoort (eds.), Kluwer Academic Publishers, Dordrecht, The Netherlands. pp. A6/1-11.

Ryden, P., Selvendran, R. R. (1990) Structural features of cell wall polysaccharides of potato (Solanum tuberosum). Carbohydr. Res. 195:257-272.

Sakurai, M. H., Kiyohara, H., Matsumoto, T., Tsumuraya, Y., Hashimoto, Y., Yamada, H. (1998) Characterization of antigenic epitopes on anti-ulcer pectic polysaccharides from Bupleurum falcatum L. using several carbohydrases. Carbohydrate Res. 311:219-229

Salehuzzaman, S. N. I. M., Jacobsen, E., Visser, R. G. F. (1992) Cloning, partial sequencing and expression of a cDNA coding for branching enzyme in cassava. Plant Mol. Biol. 20:809-819

Sambrook, J., Fritsch, E. F., Maniatis, T. 1989. Molecular cloning. A laboratory manual. Cold Spring Habor, N.Y.: Cold Spring Harbor Laboratory

Samuelsen, A. B., Lund, I., Djaromi, J. M., Paulsen, B. S., Wold, J. K., Knutsen, S. H. (1999) Structural features and anti-complementary activity of some heteroxylan polysaccharide fractions from the seeds of Plantago major L. Carbohydraten Polymers 38:135-143

Samuelsen. A B, Paulsen, B S, Wold, J K, Knutsen. S H, Yamada, H. (1998) Characterisation of a biologically active arabinogalactan from the leaves of Plantago major L. Carbohydrate Polymers 35:145-153

Satoh, S. 1998. Functions of the cell wall in the interactions of plant cells: Analysis using carrot cultured cells.—Plant Cell Physiology. 39:361-368.

Selvendran, R. R., March, J. F. & Ring, S. G. (1979) Determination of aldoses and uronic acid content of vegetable fibre Anal. Biochem. 96:282-292.

Schols, H. A., Voragen, A. G. J. (1996) Complex pectins: structure elucidation using enzymes, In Progress in Biotechnology 14 Pectins and Pectinases Eds: J. Visser and A. G. J. Voragen, Elsevier Amsterdam NL: 3-20

Sonnewald, U., Brauer, M., von Schaewen, A., Stitt, M., and Willmitzer, L. (1991). Transgenic tobacco plants expressing yeast derived invertase in either the cytosol, vacuole or apoplast: a powerful tool for studying sucrose metabolism and sink/source interactions. Plant Jour. 1:95-106.

Sørensen, S. O., Skjøt, M., Ulvskov, P., Dalbøge, H., Borkhardt, B. (1999). Modification of pectin by expression af a fungal endo-b-1,4-galactanase in potato. Plant Protein Club Workshop on Plant Proteins and the Mechanical Properties of Cell Walls, Alicante Spain. Abstract # 49

Turk, S. C. H. J., Roos, K. d., Scotti, P. A, Dun, K. v., Weisbeek, P., and Smeekens, S. C. M. (1997). The vacuolar sorting domain of sporamin transports GUS, but not levansucrase, to the plant vacuole. New phytol 136:29-38.

Visser, J., Voragen, A. G. J. (eds.) (1996) Pectins and Pectinases Progress in Biotechnology 14, Elsevier Science, Amsterdam

Visser, R. G. F. (1991) Regeneration and transformation of potato by Agrobacterium tumefaciens. Plant Tiss. Cult. Man. B5:1-9.

Visser, R. G. F., Stolte, A. &Jacobsen, E. 1991. Expression of a chimeric granule-bound starch synthase-GUS gene in transgenic potato plants.—Plant Molecular Biology. 17:691-699.

Voragen, A. G. J., Pilnik, W., Thibault, J. -F., Axelos, M. A. V. & Renard, C. M. G. C. (1995) In Food Polysaccharides and Their Applications, ed. Stephen, A. M. (Marcel Dekker, Inc.): 287-339.

Voragen, A. G. J., Schols, H. A. and Pilnik, W. Determination of the degree of methylation and acetylation of pectins by h.p.l.c. Food Hydrocolloids 1 (1), 1986, 65-70.

Wee, E., Sherrier, J. D., Prime, T., and Dupreee, P. (1998). Targeting of active sialyl-transferase to the plant golgi apparatus. Plant Cell 10:1759-1768.

Wegener, C., Bartling, S., Olsen, O., Weber, J., Wettstein, D. v. (1996) Pectate lyase in transgenic potatoes confers preactivation of defence against Erwinia carotovora. Physiol. Mol. Plant. Pathol. 49:359-376.

Weinstein, J., Lee, E. U., McEntee, K., Lai, P. H., and Paulson, J. C. (1987). Primary structure of b-galactoside alpha-2,6-sialyltransferase convesrion of membrane-bound enzyme to souluble forms by cleavage of the NH2-terminal signal anchor. J Biol. Chem. 262:17735-177743.

Yamada, H. & Kiyohara H. (1999) Complement-activating polysaccharides from medicinal herbs. In Immunomodulatory Agents from Plants, ed. Wagner, H., Birhhäuser Verlag Basel, pp 161-202

Ziegelhoffer, T., Will, J., and Austin-Phillips, S. (1999). Expression of bacterial cellulase genes in transgenic alfalfa (Medicago saliva L.). potato (Solanum tuberosum L.) and Nicotiana tabacum L.). Mol. Breed. 5:309-318.

1. A method for providing a transgenic plant material that, relative to the wild type state, is modified in a complex cell wall polysaccharide structure, the modification being at least one of the overall glycosidic linkage pattern and the monosaccharide profile, the method comprising
(i) providing a nucleic acid construct comprising a nucleotide sequence, which, following the introduction of the construct into a plant cell, results in an altered production of at least one target cell wall polysaccharide-modifying enzyme,
(ii) transforming a plant cell with the nucleic add construct, and
(iii) deriving from said transformed plant cell a plant cell culture, a plant tissue or a transgenic plant in which the production of the at least one cell wall polysaccharide-modifying enzyme is altered
to obtain transgenic plant cells, plant tissues or plants in which the targeted substrate cell wall polysaccharide, relative to the wild type plant, is modified to have a monosaccharide profile where the proportion of at least one monosaccharide is changed by at least 10% or the proportion of at least one glycosidic linkage is changed by at least 10%.

2. A method according to claim 1 wherein the nucleotide sequence, which, following the introduction of the construct into a plant cell, results in an altered production of at least one target cell wall polysaccharide-modifying enzyme is a sequence coding for such an enzyme that is capable of modifying the targeted cell wall polysaccharide.

3. A method according to claim 2 wherein the coding sequence is operably linked to a promoter directing the expression of the coding sequence.

4. A method according to claim 1 wherein the at least one target cell wall polysaccharide-modifying enzyme, the production of which is altered, is an endogenous enzyme.

5. A method according to claim 4 wherein the nucleotide sequence, which, following the introduction of the construct into a plant cell results in an altered production of at least one target cell wall polysaccharide-modifying enzyme is a sequence that modulates the expression of an endogenous sequence coding for an enzyme that is capable of modifying the targeted cell wall polysaccharide.

6. A method according to claim 5 wherein the sequence that modulates the expression of an endogenous sequence coding for an enzyme that is capable of modifying the targeted cell wall polysaccharide is a sequence coding for an antisense sequence that reduces the expression of a repressor or reduces the production of an inhibitor of the endogenous target cell wall polysaccharide-modifying enzyme.

7. A method according to claim 5 wherein the sequence that modulates the expression of an endogenous sequence coding for an enzyme that is capable of modifying the targeted cell wall polysaccharide is a sequence that, following a recombination event, causes the insertion of a new or modified promoter operably linked to the endogenous target cell wall-modifying enzyme, said promoter is capable of directing the expression of the coding sequence for the cell wall-modifying enzyme.

8. A method according to claim 4 wherein the nucleotide sequence, which, following the introduction of the construct into a plant cell results in an altered production of at least one target cell wall polysaccharide-modifying enzyme, is a sequence that, following a recombination event, causes the expression of an endogenous sequence coding for an enzyme that is capable of modifying the targeted cell wall polysaccharide to be targeted to a different location.

9. A method according to claim 2 in which the polysaccharide-modifying enzyme is of microbial or fungal origin.

10. A method according to any of claims 1-6 and 9 wherein the nucleic add construct is a viral vector that, following introduction into a plant cell, is not integrated into the genome of the cell.

11. A method according to claim 3 wherein the promoter is derived from a plant.

12. A method according to claim 11 wherein the promoter is a tissue or organ specific promoter including a storage organ specific promoter.

13. A method according to claim 12 wherein the promoter is a promoter capable of directing expression in potato tubers including a promoter selected from the group consisting of the granule bound starch synthase (GBSS) promoter and the B33 promoter.

14. A method according to any of claims 1-13 wherein the targeted complex cell wall polysaccharide is selected from the group consisting of a pectin and a hemicellulosic polysaccharide.

15. A method according to any of claims 1-14 wherein the polysaccharide-modifying enzyme is selected from the group consisting of an endo-rhamnogalacturonan hydrolase, an endo-rhamnogalacturonan lyase, an endo-galactanase, an endo-arabinanase, an arabinofuranosidase, a galactosidase such as a beta-galactosidase, a xylosidase and an exogalacturonase and an ortholog or isoform hereof.

16. A method according to any of the preceding claims wherein the nucleotide sequence, which, following the introduction of the construct into a plant cell, results in an altered production of at least one target cell wall polysaccharide-modifying enzyme, is sufficiently different from endogenous genes of the host plant so that co-suppression will not occur.

17. A method according to any of the preceding claims in which an auxiliary enzyme is co-expressed with the target cell wall polysaccharide-modifying enzyme, wherein co-expression facilitates access of the polysaccharide-modifying enzyme to its substrate.

18. A method according to claim 17 wherein the auxiliary enzyme is selected from the group consisting of an esterase, including a methyl esterase and an acetyl esterase, and a glycosidase that removes single monosaccharides from polymers, such as an arabinofuranosidase, a galactosidase, a xylosidase or a fucosidase.

19. A method for modifying the biosynthesis in a plant cell of at least one complex cell wall polysaccharide to obtain a transgenic plant material that, relative to the wild type state, is modified in a complex cell wall polysaccharide structure, the modification being at least one of the overall glycosidic linkage pattern and the monosaccharide profile, the method comprising
(i) providing a nucleic acid construct comprising a nucleotide sequence, the expression of which in a plant cell results in that at least one cell wall polysaccharide-modifying enzyme is targeted to a compartment in the plant cell where it is not normally present or in that the expression of a polypeptide naturally produced in the plant cell and affecting the biosynthesis of a cell wall polysaccharide is changed,
(ii) transforming a plant cell with the nucleic acid construct, and
(iii) deriving from said transformed plant cell a plant cell culture, a plant tissue or a transgenic plant in which the at least one cell wall polysaccharide-modifying enzyme, relative to the wild type plant, occurs in a different compartment.

20. A method according to claim 19 wherein the obtained transgenic plant cells, plant tissues or plants in which the targeted substrate cell wall polysaccharide, relative to the wild type plant, is modified to have a monosaccharide profile where the proportion of at least one monosaccharide is changed by at least 10% or the proportion of at least one glycosidic linkage is changed by at least 10%.

21. A method according to claim 19 wherein the at least one cell wall polysaccharide-modifying enzyme is targeted to the Golgi apparatus including membrane vesicles fusing with or budding off from the Golgi stacs.

22. A method according to claim 21 wherein the nucleotide sequence, the expression of which in a plant cell results in that at least one cell wall polysaccharide-modifying enzyme is targeted to the Golgi apparatus is a sequence coding for a chimeric gene product comprising the at least one cell wall polysaccharide-modifying enzyme and a sequence capable of targeting the chimeric gene product to the Golgi.

23. A method according to 22 wherein the targeting sequence is a type II membrane anchored Golgi protein or a fragment thereof or a soluble Golgi targeted protein or a fragment thereof.

24. A method according to claim 23 wherein the soluble Golgi targeted protein is Pisum sativum reversibly glycosylatable polypeptide (RGP1).

25. A method according to claim 23 wherein the type II membrane anchored Golgi protein is selected from the group consisting of a sialyl transferase, a N-acetylglucosaminyltransferase, a fucosyl transferase, a xylosyl transferase and a galactosyl transferase.

26. A method according to any of claims 19-25 wherein the nucleotide sequence, the expression of which in a plant cell results in that at least one cell wall polysaccharide-modifying enzyme is targeted to a compartment in the plant cell where it is not normally present, is operably linked to a promoter in the genome of the plant cell into which it is introduced.

27. A method for providing a transgenic plant comprising parts in which at least one complex cell wall polysaccharide can be enzymatically processed after harvest by an enzyme present in the plant material itself, the method comprising
(i) providing a nucleic acid construct comprising a nucleotide sequence, which, following the introduction of the construct into a plant cell, causes a cell wall polysaccharide-modifying enzyme to be expressed in a non-apoplastic or non-Golgi compartment or the expression of a cell wall polysaccharide-modifying enzyme in a form that is inactive under in vivo conditions but can be activated after harvest of plant material derived from the plant cell,
(ii) transforming a plant cell with the nucleic acid construct, and
(iii) deriving from said transformed plant cell a transgenic plant material in which, under appropriate post harvest conditions, the at least one complex cell wall polysaccharide can be enzymatically processed after harvest by bringing the at least one complex cell wall polysaccharide into contact with the cell wall polysaccharide-modifying enzyme that is expressed in a non-apoplastic or non-Golgi compartment or by subjecting the harvested plant material to conditions under which the enzyme being expressed in an in vivo inactive form is activated.

28. A method according to claim 27 wherein the nucleotide sequence, which, following the introduction of the nucleic acid construct into a plant cell, causes a cell wall polysaccharide-modifying enzyme to be expressed in a non-apoplastic or non-Golgi compartment is a sequence causing the cell wall polysaccharide-modifying enzyme to be targeted during growth of the plant to a cell compartment selected from the group consisting of a vacuole, the endoplasmic reticulum, the cytoplasm and a plastid.

29. A method according to claim 28 wherein the cell wall polysaccharide-modifying enzyme caused to be expressed in a non-apoplastic or non-Golgi compartment is encoded by a sequence comprised in the nucleic add construct that is introduced into the plant cell.

30. A method according to claim 28 wherein the cell wall polysaccharide-modifying enzyme caused to be expressed in a non-apoplastic or non-Golgi compartment is encoded by an endogenous sequence present in the genome of the cell into which the nucleic acid construct is introduced.

31. A method according to any of claims 27-30 wherein the cell wall polysaccharide-modifying enzyme is selected from the group consisting of an endo-polygalacturonase, an endo-pectin lyase, a pectate lyase, a rhamnogalacturonan hydrolase, a rhamnogalacturonan lyase, an endo-glucanase, an endo-xylanase and an isoform or ortholog hereof.

32. A method according to any of claims 27-31 wherein expression of the cell wall polysaccharide-modifying enzyme is directed by a plant promoter.

33. A method according to any of claims 27-32 wherein the at least one complex cell wall polysaccharide that can be enzymatically processed after harvest by an enzyme present in the plant material itself is selected from the group consisting of a pectin and a hemicellulosic polysaccharide.

34. A method according to claim 33 wherein the post harvest processing of pectin is in the regions between rhamnogalacturonan and homogalacturonan regions.

35. A method according to any of claims 27-34 wherein the plant cell is further transformed with a nucleic acid sequence causing an enzyme that is capable of in vivo modifying the structure of at least one complex cell wall polysaccharide including a cell wall polysaccharide that can be enzymatically processed after harvest by an enzyme present in the plant material itself to be expressed.

36. A method according to claim 35 wherein the nucleic add sequence causing an enzyme that is capable of in vivo modifying the structure of at least one complex cell wall polysaccharide to be expressed is a sequence coding for a cell wall polysaccharide-modifying enzyme.

37. A method according to claim 36 wherein the nucleic acid sequence causing an enzyme that is capable of in vivo modifying the structure of at least one complex cell wall polysaccharide is a sequence coding for a product that affects the expression of an endogenous sequence coding for a cell wall polysaccharide-modifying enzyme.

38. A method according to claim 36 or 37 wherein the cell wall polysaccharide-modifying enzyme is targeted to the apoplast.

39. A method according to any of claims 35-38 wherein the enzyme that is capable of in vivo modifying the structure of at least one complex cell wall polysaccharide is selected from the group consisting of an endo-rhamnogalacturonan hydrolase, an endo-rhamnogalacturonan lyase, an endo-galactanase, an endo-arabinanase, an arabinofuranosidase, a galactosidase such as a beta-galactosidase, a xylosidase and an exo-galacturonase and an ortholog or isoform hereof.

40. A method of providing a plant cell wall polysaccharide material that, relative to the wild type state, is modified in a complex cell wall polysaccharide structure, the modification being at least one of the overall glycosidic linkage pattern and the monosaccharide profile, the method comprising:
(i) providing transgenic plants using a method according to any of claims 28-39,
(ii) cultivating and harvesting said plants and isolating herefrom parts in which at least one complex cell wall polysaccharide can be enzymatically processed after harvest by an enzyme present in the plant material itself,
(iii) subjecting said parts to conditions where the cell wall polysaccharide-modifying enzyme expressed in a non-apoplastic or non-Golgi compartment is brought into contact with its cell wall polysaccharide substrate or the cell wall polysaccharide-modifying enzyme expressed in a form that is inactive under in vivo conditions becomes activated to obtain a modified cell wall polysaccharide, and
(iv) isolating the modified cell wall polysaccharide material.

41. A method according to claim 40 wherein the obtained modified cell wall polysaccharide in the material as obtained is modified to have a monosaccharide profile where the proportion of at least one monosaccharide is changed by at least 10% or the proportion of at least one glycosidic linkage is changed by at least 10%.

42. A method according to claim 40 or 41 wherein the plant parts in which at least one complex cell wall polysaccharide is enzymatically processed after harvest by an enzyme present in the plant material itself is a potato plant part.

43. A method according to any of claims 39-42 wherein the cell wall polysaccharide that is post harvest modified is pectin.

44. A plant cell wall polysaccharide material having, relative to the wild type state, a modified structure and composition, said material is obtained by the method of any of claims 39-43.

45. A transgenic plant or progeny of the plant or part thereof obtained by the method of any of claims 1-18.

46. A plant cell wall polysaccharide-containing material that, relative to the wild type state, is modified in a complex cell wall polysaccharide structure, the modification being at least one of the overall glycosidic linkage pattern and the monosaccharide profile, said plant cell wall polysaccharide-containing material is obtained from the transgenic plant or progeny or part according to claim 45.

47. A plant cell wall polysaccharide-containing material according to claim 46 which, relative to a material containing the corresponding wild type cell wall polysaccharide, has at least one altered functional characteristic.

48. A plant cell wall polysaccharide-containing material according to claim 47 where the altered functional characteristic is selected from the group consisting of pharmaceutical activity, water binding capacity, processibility, gelling property, thickening property and digestibility.

49. Use of the transgenic plant or progeny of the plant or part thereof according to claim 45 or the plant cell wall polysaccharide-containing material according to any of claims 46-48 in the manufacturing of a product selected from the group consisting of a food product, a feed product, a pharmaceutical or medical product and a cosmetic products.

50. A pharmaceutical or medical product comprising a plant cell wall polysaccharide-containing material according to any of claims 46-48, including a product selected from the group consisting of a pharmaceutical composition, an implant material, a medical device, a wound dressing and a surgical adhesive.

51. A transgenic plant or progeny of the plant or part thereof obtained by the method according to any of claims 19-39.

52. A plant cell wall polysaccharide-containing material that, relative to the wild type state, is modified in a complex cell wall polysaccharide structure, the modification being at least one of the overall glycosidic linkage pattern and the monosaccharide profile, said plant cell wall polysaccharide-containing material is obtained from the transgenic plant or progeny or part according to claim 51.

53. A plant cell wall polysaccharide-containing material according to claim 52 which, relative to a material containing the corresponding wild type cell wall polysaccharide, has at least one altered functional characteristic.

54. A plant cell wall polysaccharide-containing material according to claim 53 where the altered functional characteristic is selected from the group consisting of pharmaceutical activity, water binding capacity, processibility, gelling property, thickening property and digestibility.

55. Use of the transgenic plant or progeny of the plant or part thereof according to claim 51 or the plant cell wall polysaccharide-containing material according to any of claims 52-54 in the manufacturing of a product selected from the group consisting of a food product, a feed product, a pharmaceutical or medical product and a cosmetic products.

56. A pharmaceutical or medical product comprising a plant cell wall polysaccharide-containing material according to any of claims 52-54, including a product selected from the group consisting of a pharmaceutical composition, an implant material, a medical device, a wound dressing and a surgical adhesive.

57. A method of producing a material comprising a complex plant cell wall polysaccharide that, relative to the wild type state, is modified in a complex cell wall polysaccharide structure, the modification being at least one of the overall glycosidic linkage pattern and the monosaccharide profile, the method comprising
(i) providing a cultivatable transgenic plant using a method according to any of claims 1-18 and 19-39 or a cultivatable progeny hereof,
(ii) cultivating said transgenic plant or progeny under appropriate plant cultivation conditions to obtain a plant material comprising at least one complex cell wall polysaccharidethat, relative to the wild type state, is modified in a complex cell wall polysaccharide structure, the modification being at least one of the overall glycosidic linkage pattern and the monosaccharide profile, and
(iii) isolating from the cultivated plants the material comprising the modified cell wall polysaccharide.

58. A method according to claim 57 wherein the plant or progeny that is cultivated is a potato plant.

59. A method according to claim 57 or 58 wherein the material isolated from the cultivated plants comprises a cell wall polysaccharide that, relative to the wild type plant, is modified to have a monosaccharide profile where the proportion of at least one monosaccharide is changed by at least 10% or the proportion of at least one glycosidic linkage is changed by at least 10%.
